(19) United States
(12) Patent Application Publication
(10) Pub. No.: US 2013/0131106 A1
(43) Pub. Date: May 23, 2013

(54) BIFUNCTIONAL RHO KINASE INHIBITOR COMPOUNDS, COMPOSITION AND USE

(71) Applicant: INSPIRE PHARMACEUTICALS, INC., Whitehouse Station, NJ (US)

(72) Inventors: John W. LAMPE, Dedham, MA (US); Paul S. WATSON, Carrboro, NC (US); Jason L. VITTITOW, Flanders, NJ (US); Ward M. PETERSON, Morrisville, NC (US)

(73) Assignee: INSPIRE PHARMACEUTICALS, INC., Whitehouse Station, NJ (US)

(21) Appl. No.: 13/742,073

(22) Filed: Jan. 15, 2013

Related U.S. Application Data
(63) Continuation of application No. PCT/US2011/044148, filed on Jul. 15, 2011.
(60) Provisional application No. 61/365,681, filed on Jul. 19, 2010.

(51) Int. Cl. C07D 401/12; C07D 401/14

(52) U.S. Cl. CPC C07D 401/12; C07D 401/14
USPC 514/310; 546/143; 546/199; 514/322

(57) ABSTRACT

This invention relates to synthetic bifunctional compounds comprising a first rho-associated kinase (ROCK) inhibiting compound and a second pharmaceutically active compound with complementary activity; the first and the second compounds are covalently linked by a biologically labile bond. This invention also relates to methods of making such compounds. The invention also relates to methods of using such bifunctional compounds in the prevention or treatment of diseases or conditions that are affected or can be assisted by altering the integrity or rearrangement of the cytoskeleton. Particularly, this invention relates to methods of treating ophthalmic diseases such as disorders in which intraocular pressure is elevated, for example primary open-angle glaucoma, using the bifunctional compounds.

This application is a continuation of PCT/US2011/044148, filed Jul. 15, 2011; which claims the priority of U.S. Provisional Application No. 61/365,681, filed Jul. 19, 2010. The contents of the above-identified applications are incorporated herein by reference in their entireties.

### TECHNICAL FIELD

This invention relates to synthetic bifunctional compounds comprising a first rho-associated kinase (ROCK) inhibiting compound covalently linked to a second pharmaceutically active compound by a biologically labile bond. The second active compound is pilocarpine, a prostaglandin, or a derivative thereof. The invention also relates to methods of using such bifunctional compounds for treating ophthalmic diseases such as disorders in which intraocular pressure is elevated, for example primary open-angle glaucoma.

### BACKGROUND OF THE INVENTION

The Rho family of small GTP binding proteins can be activated by several extracellular stimuli such as growth factors, hormones and mechanic stress and function as a molecular signaling switch by cycling between an inactive GDP-bound form and an active GTP-bound form to elicit cellular responses. Rho kinase (ROCK) functions as a key downstream mediator of Rho and exists as two isoforms (ROCK 1 and ROCK 2) that are ubiquitously expressed. ROCKs are serine/threonine kinases that regulate the function of a number of substrates including cytoskeletal proteins such as adducin, moesin, Na⁺—H⁺ exchanger 1 (NHE1), LIM-kinase and vimentin, contractile proteins such as the myosin light chain phosphatase binding subunit (MYPT-1), CPI-17, myosin light chain and calponin, microtubule associated proteins such as Tau and MAP-2, neuronal growth cone associate proteins such as CRMP-2, signaling proteins such as PTEN and transcription factors such as serum response factor (Loirand et al, Circ Res 98:322-334 (2006)). ROCK is also required for cellular transformation induced by RhoA. As a key intermediary of multiple signaling pathways, ROCK regulates a diverse array of cellular phenomena including cytoskeletal rearrangement, actin stress fiber formation, proliferation, chemotaxis, cytokinesis, cytokine and chemokine secretion, endothelial or epithelial cell junction integrity, apoptosis, transcriptional activation and smooth muscle contraction. As a result of these cellular actions, ROCK regulates many physiologic processes such as vasoconstriction, bronchoconstriction, tissue remodeling, inflammation, edema, platelet aggregation and proliferative disorders.

One well documented example of ROCK activity is in smooth muscle contraction. In smooth muscle cells ROCK mediates calcium sensitization and smooth muscle contraction. Agonists (noradrenaline, acetylcholine, endothelin, etc.) that bind to G protein coupled receptors produce contraction by increasing both the cytosolic Ca²⁺ concentration and the Ca²⁺ sensitivity of the contractile apparatus. The Ca²⁺-sensitizing effect of smooth muscle constricting agents is ascribed to ROCK-mediated phosphorylation of MYPT-1, the regulatory subunit of myosin light chain phosphatase (MLCP), which inhibits the activity of MLCP resulting in enhanced phosphorylation of the myosin light chain and smooth muscle contraction (WO 2005/003101A2, WO 2005/034866A2).

ROCK inhibitors have utility in treating many disorders. One example is the treatment of ophthalmic diseases such as glaucoma, allergic conjunctivitis, macular edema and degeneration, and blepharitis.

Glaucoma is an ophthalmic disease that leads to irreversible visual impairment. It is the fourth most common cause of blindness and the second most common cause of visual loss in the United States, and the most common cause of irreversible visual loss among African-Americans. Generally speaking, the disease is characterized by a progressive optic neuropathy caused at least in part by deleterious effects resulting from increased intraocular pressure. In normal individuals, intraocular pressures range from 12 to 20 mm Hg, averaging approximately 16 mm Hg. However, in individuals suffering from primary open angle glaucoma, intraocular pressures generally rise above 22 to 30 mm Hg. In angle closure or acute glaucoma, intraocular pressure can reach as high as 70 mm Hg leading to blindness within only a few days. Typical treatments for glaucoma comprise a variety of pharmaceutical approaches for reducing intraocular pressure (IOP), but each with some drawbacks. Beta-blockers and carbonic anhydrase inhibitors reduce aqueous humor production, which is needed to nourish the avascular lens and corneal endothelial cells. Prostaglandins affect the uvealscleral outflow pathway, which only accounts for 10% of the total outflow facility. There are currently no commercially approved therapeutic agents which act directly upon the trabecular meshwork, the site of aqueous humor drainage where increased resistance to aqueous humor outflow is responsible for elevated TOP.

The most common allergic eye disease, allergic conjunctivitis (AC) can be subdivided into acute, seasonal and perennial. All three types result from classic Type I IgE-mediated hypersensitivity (Abelson, M B., et. al. Sury Ophthalmol; 38(S):115, 1993). Allergic conjunctivitis is a relatively benign ocular disease of young adults (average age of onset of 20 years of age) that causes significant suffering and use of healthcare resources, although it does not threaten vision. Ocular allergy is estimated to affect 20 percent of the population on an annual basis, and the incidence is increasing (Abelson, M B et. al., Surv. Ophthalmol., 38(S):115, 1993). AC impacts productivity and while there are a variety of agents available for the treatment of AC, numerous patients still lack good control of symptoms and some are tolerating undesired side effects. Surveys have shown 20% of patients with AC are not fully satisfied with their AC medications and almost 50% feel they receive insufficient attention from their physicians (Mahr, et al., Allergy Asthma Proc, 28(4):404-9, 2007).

Macular edema is a condition that occurs when damaged (or newly formed) blood vessels leak fluid onto the macula, a critical part of the retina for visual acuity, causing it to swell and blur vision. Macular edema is a common problem in diabetic retinopathy, where retinal vessel injury causes edema. Edema also occurs in the proliferative phase of diabetic retinopathy, when newly formed vessels leak fluid into either, or both, the macula and/or vitreous. Macular edema is commonly problematic in age-related macular degeneration (wet form) as well, where newly formed capillaries (angiogenesis) leak fluid into the macula. Age related macular degeneration (AMD) is a progressive eye condition affecting as many as 10 million Americans. AMD is the number one cause of vision loss and legal blindness in adults over 60 in the U.S. As the population ages, and the “baby boomers” advance into their 60's and 70's, a virtual epidemic of AMD will be prevalent. The disease affects the macula of the eye, where the sharpest central vision occurs. Although it rarely results in complete blindness, it robs the individual of all but the outermost, peripheral vision, leaving only dim images or black holes at the center of vision.

Blepharitis, also known as Lid Margin Disease (LMD), is a non-contagious inflammation of the eyelids that manifests itself through scaling and flaking around the eyelashes, excess sebum production and oily scaly discharge, mucopurulent discharge, and matted, hard crusts around the lashes. Accumulation of crust, discharge or debris on the eyelashes and lid margins creates an ideal environment for overgrowth of the staphylococcal bacteria naturally found on the skin of the eyelids and increases the chance of infection, allergic reaction and tear break down. Blepharitis disturbs the production of the critical, outer lipid layer of the tear film which causes the entire tear to evaporate, resulting in dry eye. A reduced tear quantity doesn't properly dilute bacteria and irritants, nor wash inflammatory products away from the lashes and lid margin, so they accumulate and lead to further inflammation worsening the cycle of disease, with blepharitis, meibomian gland dysfunction and dry eye perpetuating each other.

Because of the need to use multiple pharmaceutical agents to manage ophthalmic diseases, there exists a need for a single agent that combines ROCK inhibition with adjunct pharmacologic activity in a convenient, well-tolerated dosage form.

### SUMMARY OF THE INVENTION

The present invention is directed to a compound of Formula III, which comprises a rho kinase inhibitor covalently linked to a prostaglandin or pilocarpine, or derivatives thereof. The covalent linkage is metabolically labile, which allows for said compound to break apart into its constituitive pieces upon administration to a subject, thus providing an additive or synergistic effect of each constituitive piece. The present invention is also directed to a pharmaceutical composition comprising such compound and a pharmaceutically acceptable carrier.

The present invention is further directed to a method of preventing or treating ophthalmic diseases or conditions associated with cellular relaxation and/or changes in cell-substratum adhesions. The invention particularly provides a method of reducing intraocular pressure, including treating glaucoma such as primary open-angle glaucoma. The methods comprise the steps of identifying a subject in need of treatment, and administering to the subject a compound of Formula III, in an amount effective to treat the disease.

The active compound is delivered to a subject by systemic administration or local administration.

### DETAILED DESCRIPTION OF THE INVENTION

### Definitions

When present, unless otherwise specified, the following terms are generally defined as, but are not limited to, the following:

Halo substituents are taken from fluorine, chlorine, bromine, and iodine.

“Alkyl” refers to groups of from 1 to 12 carbon atoms inclusively, either straight chained or branched, more preferably from 1 to 8 carbon atoms inclusively, and most preferably 1 to 6 carbon atoms inclusively.

“Alkenyl” refers to groups of from 2 to 12 carbon atoms inclusively, either straight or branched containing at least one double bond but optionally containing more than one double bond.

“Alkynyl” refers to groups of from 2 to 12 carbon atoms inclusively, either straight or branched containing at least one triple bond but optionally containing more than one triple bond, and additionally optionally containing one or more double bonded moieties.

“Alkoxy” refers to the group alkyl-O— wherein the alkyl group is as defined above including optionally substituted alkyl groups as also defined above.

“Alkenoxy” refers to the group alkenyl-O— wherein the alkenyl group is as defined above including optionally substituted alkenyl groups as also defined above.

“Alkynoxy” refers to the group alkynyl-O— wherein the alkynyl group is as defined above including optionally substituted alkynyl groups as also defined above.

“Aryl” refers to an unsaturated aromatic carbocyclic group of from 6 to 14 carbon atoms inclusively having a single ring (e.g., phenyl) or multiple condensed rings (e.g., naphthyl or anthryl). Preferred aryls include phenyl, naphthyl and the like.

“Arylalkyl” refers to aryl-alkyl- groups preferably having from 1 to 6 carbon atoms inclusively in the alkyl moiety and from 6 to 10 carbon atoms inclusively in the aryl moiety. Such arylalkyl groups are exemplified by benzyl, phenethyl and the like.

“Arylalkenyl” refers to aryl-alkenyl- groups preferably having from 2 to 6 carbon atoms in the alkenyl moiety and from 6 to 10 carbon atoms inclusively in the aryl moiety.

“Arylalkynyl” refers to aryl-alkynyl- groups preferably having from 2 to 6 carbon atoms inclusively in the alkynyl moiety and from 6 to 10 carbon atoms inclusively in the aryl moiety.

“Cycloalkyl” refers to cyclic alkyl groups of from 3 to 12 carbon atoms inclusively having a single cyclic ring or multiple condensed rings which can be optionally substituted with from 1 to 3 alkyl groups. Such cycloalkyl groups include, by way of example, single ring structures such as cyclopropyl, cyclobutyl, cyclopentyl, cyclooctyl, 1-methylcyclopropyl, 2-methylcyclopentyl, 2-methylcyclooctyl, and the like, or multiple ring structures such as adamantyl, and the like.

“Cycloalkenyl” refers to cyclic alkenyl groups of from 4 to 12 carbon atoms inclusively having a single cyclic ring or multiple condensed rings and at least one point of internal unsaturation, which can be optionally substituted with from 1 to 3 alkyl groups. Examples of suitable cycloalkenyl groups include, for instance, cyclobut-2-enyl, cyclopent-3-enyl, cyclooct-3-enyl and the like.

“Cycloalkylalkyl” refers to cycloalkyl-alkyl- groups preferably having from 1 to 6 carbon atoms inclusively in the alkyl moiety and from 6 to 10 carbon atoms inclusively in the cycloalkyl moiety. Such cycloalkylalkyl groups are exemplified by cyclopropylmethyl, cyclohexylethyl and the like.

“Cycloalkylalkenyl” refers to cycloalkyl-alkenyl- groups preferably having from 2 to 6 carbon atoms inclusively in the alkenyl moiety and from 6 to 10 carbon atoms inclusively in the cycloalkyl moiety. Such cycloalkylalkenyl groups are exemplified by cyclohexylethenyl and the like.

“Cycloalkylalkynyl” refers to cycloalkyl-alkynyl- groups preferably having from 2 to 6 carbon atoms inclusively in the alkynyl moiety and from 6 to 10 carbon atoms inclusively in the cycloalkyl moiety. Such cycloalkylalkynyl groups are exemplified by cyclopropylethynyl and the like.

“Heteroaryl” refers to a monovalent aromatic heterocyclic group of from 1 to 10 carbon atoms inclusively and 1 to 4 heteroatoms inclusively selected from oxygen, nitrogen and sulfur within the ring. Such heteroaryl groups can have a single ring (e.g., pyridyl or furyl) or multiple condensed rings (e.g., indolizinyl or benzothienyl).

“Heteroarylalkyl” refers to heteroaryl-alkyl- groups preferably having from 1 to 6 carbon atoms inclusively in the alkyl moiety and from 6 to 10 atoms inclusively in the heteroaryl moiety. Such heteroarylalkyl groups are exemplified by pyridylmethyl and the like.

“Heteroarylalkenyl” refers to heteroaryl-alkenyl- groups preferably having from 2 to 6 carbon atoms inclusively in the alkenyl moiety and from 6 to 10 atoms inclusively in the heteroaryl moiety.

“Heteroarylalkynyl” refers to heteroaryl-alkynyl- groups preferably having from 2 to 6 carbon atoms inclusively in the alkynyl moiety and from 6 to 10 atoms inclusively in the heteroaryl moiety.

“Heterocycle” refers to a saturated or unsaturated group having a single ring or multiple condensed rings, from 1 to 8 carbon atoms inclusively and from 1 to 4 hetero atoms inclusively selected from nitrogen, sulfur or oxygen within the ring. Such heterocyclic groups can have a single ring (e.g., piperidinyl or tetrahydrofuryl) or multiple condensed rings (e.g., indolinyl, dihydrobenzofuran or quinuclidinyl). Preferred heterocycles include piperidinyl, pyrrolidinyl and tetrahydrofuryl.

“Heterocycle-alkyl” refers to heterocycle-alkyl- groups preferably having from 1 to 6 carbon atoms inclusively in the alkyl moiety and from 6 to 10 atoms inclusively in the heterocycle moiety. Such heterocycle-alkyl groups are exemplified by morpholino-ethyl, pyrrolidinylmethyl, and the like.

“Heterocycle-alkenyl” refers to heterocycle-alkenyl- groups preferably having from 2 to 6 carbon atoms inclusively in the alkenyl moiety and from 6 to 10 atoms inclusively in the heterocycle moiety.

“Heterocycle-alkynyl” refers to heterocycle-alkynyl- groups preferably having from 2 to 6 carbon atoms inclusively in the alkynyl moiety and from 6 to 10 atoms inclusively in the heterocycle moiety.

Examples of heterocycles and heteroaryls include, but are not limited to, furan, thiophene, thiazole, oxazole, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, pyrrolidine, indoline and the like.

Unless otherwise specified, positions occupied by hydrogen in the foregoing groups can be further substituted with substituents exemplified by, but not limited to, hydroxy, oxo, nitro, methoxy, ethoxy, alkoxy, substituted alkoxy, trifluoromethoxy, haloalkoxy, fluoro, chloro, bromo, iodo, halo, methyl, ethyl, propyl, butyl, alkyl, alkenyl, alkynyl, substituted alkyl, trifluoromethyl, haloalkyl, hydroxyalkyl, alkoxyalkyl, thio, alkylthio, acyl, carboxy, alkoxycarbonyl, carboxamido, substituted carboxamido, alkylsulfonyl, alkylsulfinyl, alkylsulfonylamino, sulfonamido, substituted sulfonamido, cyano, amino, substituted amino, alkylamino, dialkylamino, aminoalkyl, acylamino, amidino, amidoximo, hydroxamoyl, phenyl, aryl, substituted aryl, aryloxy, arylalkyl, arylalkenyl, arylalkynyl, pyridyl, imidazolyl, heteroaryl, substituted heteroaryl, heteroaryloxy, heteroarylalkyl, heteroarylalkenyl, heteroarylalkynyl, cyclopropyl, cyclobutyl, cyclopentyl, cyclohexyl, cycloalkyl, cycloalkenyl, cycloalkylalkyl, substituted cycloalkyl, cycloalkyloxy, pyrrolidinyl, piperidinyl, morpholino, heterocycle, (heterocycle)oxy, and (heterocycle)alkyl; and preferred heteroatoms are oxygen, nitrogen, and sulfur. It is understood that where open valences exist on these substituents they can be further substituted with alkyl, cycloalkyl, aryl, heteroaryl, and/or heterocycle groups, that where these open valences exist on carbon they can be further substituted by halogen and by oxygen-, nitrogen-, or sulfur-bonded substituents, and where multiple such open valences exist, these groups can be joined to form a ring, either by direct formation of a bond or by formation of bonds to a new heteroatom, preferably oxygen, nitrogen, or sulfur. It is further understood that the above subtitutions can be made provided that replacing the hydrogen with the substituent does not introduce unacceptable instability to the molecules of the present invention, and is otherwise chemically reasonable.

The term “heteroatom-containing substituent” refers to substituents containing at least one non-halogen heteroatom. Examples of such substituents include, but are not limited to, hydroxy, oxo, nitro, methoxy, ethoxy, alkoxy, substituted alkoxy, trifluoromethoxy, haloalkoxy, hydroxyalkyl, alkoxyalkyl, thio, alkylthio, acyl, carboxy, alkoxycarbonyl, carboxamido, substituted carboxamido, alkylsulfonyl, alkylsulfinyl, alkylsulfonylamino, sulfonamido, substituted sulfonamido, cyano, amino, substituted amino, alkylamino, dialkylamino, aminoalkyl, acylamino, amidino, amidoximo, hydroxamoyl, aryloxy, pyridyl, imidazolyl, heteroaryl, substituted heteroaryl, heteroaryloxy, heteroarylalkyl, heteroarylalkenyl, heteroarylalkynyl, cycloalkyloxy, pyrrolidinyl, piperidinyl, morpholino, heterocycle, (heterocycle)oxy, and (heterocycle)alkyl; and preferred heteroatoms are oxygen, nitrogen, and sulfur. It is understood that where open valences exist on these substituents they can be further substituted with alkyl, cycloalkyl, aryl, heteroaryl, and/or heterocycle groups, that where these open valences exist on carbon they can be further substituted by halogen and by oxygen-, nitrogen-, or sulfur-bonded substituents, and where multiple such open valences exist, these groups can be joined to form a ring, either by direct formation of a bond or by formation of bonds to a new heteroatom, preferably oxygen, nitrogen, or sulfur. It is further understood that the above subtitutions can be made provided that replacing the hydrogen with the substituent does not introduce unacceptable instability to the molecules of the present invention, and is otherwise chemically reasonable.

“Pharmaceutically acceptable salts” are salts that retain the desired biological activity of the parent compound and do not impart undesired toxicological effects. Pharmaceutically acceptable salt forms include various polymorphs as well as the amorphous form of the different salts derived from acid or base additions. The acid addition salts can be formed with inorganic or organic acids. Illustrative but not restrictive examples of such acids include hydrochloric, hydrobromic, sulfuric, phosphoric, citric, acetic, propionic, benzoic, napthoic, oxalic, succinic, maleic, fumaric, malic, adipic, lactic, tartaric, salicylic, methanesulfonic, 2-hydroxyethanesulfonic, toluenesulfonic, benzenesulfonic, camphorsulfonic, and ethanesulfonic acids. The pharmaceutically acceptable base addition salts can be formed with metal or organic counterions and include, but are not limited to, alkali metal salts such as sodium or potassium; alkaline earth metal salts such as magnesium or calcium; and ammonium or tetraalkyl ammonium salts, i.e., NX₄⁺ (wherein X is C₁₋₄).

“Tautomers” are compounds that can exist in one or more forms, called tautomeric forms, which can interconvert by way of a migration of one or more hydrogen atoms in the compound accompanied by a rearrangement in the position of adjacent double bonds. These tautomeric forms are in equilibrium with each other, and the position of this equilibrium will depend on the exact nature of the physical state of the compound. It is understood that where tautomeric forms are possible, the current invention relates to all possible tautomeric forms.

“Solvates” are addition complexes in which a compound of the invention is combined with a pharmaceutically acceptable cosolvent in some fixed proportion. Cosolvents include, but are not limited to, water, methanol, ethanol, 1-propanol, isopropanol, 1-butanol, isobutanol, tert-butanol, acetone, methyl ethyl ketone, acetonitrile, ethyl acetate, benzene, toulene, xylene(s), ethylene glycol, dichloromethane, 1,2-dichloroethane, N-methylformamide, N,N-dimethylformamide, N-methylacetamide, pyridine, dioxane, and diethyl ether. Hydrates are solvates in which the cosolvent is water. It is to be understood that the definitions of compounds of the invention encompass all possible hydrates and solvates, in any proportion, which possess the stated activity.

“An effective amount” is the amount effective to treat a disease by ameliorating the pathological condition or reducing the symptoms of the disease. “An effective amount” is the amount effective to improve at least one of the parameters relevant to measurement of the disease.

### Bifunctional Rho Kinase Inhibitor Compounds

The present invention is directed to a bifunctional compound, in which a rho kinase inhibitor compound is covalently linked to a second pharmaceutically active compound. The ROCK inhibitor compound and the second pharmaceutically active compound have complementary activities and have similar dosage requirements. The covalent linkage is metabolically labile, which allows for said compound to break apart into the ROCK inhibitor compound and the second compound upon administration to a subject, thus often providing an additive or synergistic effect of each active agent. The bifunctional compound is useful when co-delivery of the two agents (rho kinase inhibitor and the second drug) is advantageous.

Because of the need to use multiple pharmaceutical agents to manage ophthalmic disease, such as glaucoma, having therapies that achieve the effect of multiple mechanistic approaches in a single agent is advantageous. A single therapeutic agent allows for better patient compliance.

The bifunctional rho kinase inhibitor compounds useful for this invention include compounds of general Formula I, tautomers, pharmaceutically-acceptable salts, solvates, and/or hydrates thereof.

### Formula I

Compounds of Formula I are as follows:

Drug₂(FG₂)-Link-Drug₁(FG₁) Formula I

wherein Drug₁(FG₁) is a rho kinase inhibitor compound containing a functional group FG₁;

Drug₂(FG₂) is a second drug containing a functional group FG₂. Drug₂ is selected from the prostaglandin F_{2α} agonists and derivatives of the muscarinic agonist pilocarpine.

FG₁ and FG₂ are independently functional groups on Drug₁ and Drug₂, respectively. FG₁ is a functional group capable of participating in the formation of biologically labile bonds, including hydroxyl, carboxylic acid, primary amine, secondary amine, tertiary amine heterocyclic nitrogen, heteroaryl nitrogen, and primary or secondary sulfonamide.

FG₂ is a carboxylic acid or ester, —OC(O)—.

Link is a connecting unit which forms biologically labile bonds with FG₁ and FG₂. Link is selected from the following specific groups:

1. Link-1: Absent

2. Link-2:

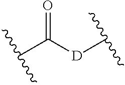

3. Link-3:

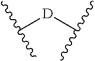

4. Link-4:

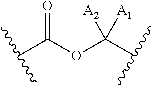

wherein A₁ and A₂ are independently hydrogen, lower alkyl (C₁₋₆ alkyl), or arylalkyl, optionally substituted, and A₁ and A₂ are optionally joined to form a ring through a direct bond or through a bond to a nitrogen, oxygen, or sulfur atom;

D is alkyl, alkenyl, aryl, arylalkyl, arylalkenyl, heteroaryl, heteroarylalkyl, heteroarylalkenyl, cycloalkyl, cycloalkenyl, cycloalkylalkyl, cycloalkylalkenyl, heterocycle, (heterocycle)alkyl, or (heterocycle)alkenyl, optionally substituted.

One skilled in the art will recognize that some specific combinations of the Link groups 1-4 with functional groups FG₁ and FG₂ are more useful in forming biologically labile bonds than others. A preferred combination is the use of Link-1 or Link-2 in cases in which FG₂ is a carboxylic acid, and FG₁ is an alcohol, allowing the formation of one or two ester bonds.

Additional preferred combinations are the use of Link-3 in the case where both FG₁ and FG₂ are carboxylic acids allowing the formation of two ester bonds, or in the case where FG₁ is a tertiary amine and FG₂ is a carboxylic acid allowing the formation of an ester-methylene-ammonium linkage. Linkage 3 is also useful in the case FG₁ is a non-basic or weakly basic nitrogen bearing a hydrogen in a heterocyclic or heteroaryl ring, such as imidazole, pyrazole, or tetrazole, or in the case where FG₁ is a functional group with a nitrogen bearing a moderately acidic hydrogen, such as an acylsulfonamide or sulfonyl aniline.

Additional preferred combinations are the use of Link-4 in the case where FG₁ is a primary or secondary amine.

The groups A₁, A₂, and D can be selected in such a way as to optimize the pharmaceutical properties of the resulting compound of Formula I. Specifically, modifications in these groups can be made to alter the lipophilicity, hydrophilicity, crystallinity, and other properties of the Formula I compound. These changes can be used to optimize the solubility of the compounds, the formulation for delivery, or the conversion into respirable particles. Further, these changes can be used to adjust the permeability of these compounds with respect to target biological tissues. Additionally, structural changes can be made in such a way as to optimize the rate at which the compound is converted in vivo into its two components, i.e., two therapeutically active subunits. In one application of these structural changes, the groups A₁, A₂, and D can be selected in such a way as to encourage the formation of micelles or vesicles containing the formulated Formula I compound as way to delay the release of the component subunits. The structural changes described above can be made without altering the fundamental therapeutic value of the component subunits.

Preferred A₁ and A₂ are independently hydrogen, methyl, and ethyl. Preferred D includes phenyl, pyridyl, (CH₂)ᵢCHA₃(CH₂)ⱼ, and (CH₂)ᵢC₆H₄(CH₂)ⱼ, where i and j are independently 0-4 inclusive, and A₃ is hydrogen, alkyl, aryl, arylalkyl, heteroaryl, heteroarylalkyl, cycloalkyl, or cycloalkylalkyl.

Specifically, in the group Link-2, the most preferred D is CH₂ or CHCH₃. For Link-3, the most preferred D is CH₂, CH(CH₃), (CH₂)₃, (CH₂)₄, (CH₂)₅, and (CH₂)₂CHCH₃. For Link-4, the most preferred A₁ is hydrogen and the most preferred A₂ is hydrogen and methyl.

In a preferred embodiment of the invention, Drug₁(FG₁) of Formula I is a rho kinase inhibitor compound as disclosed in Formula II of US2008/0214614A1. Specifically, in this embodiment, Drug₁(FG₁) is a compound of Formula II:

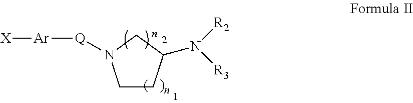

wherein:
Q is C═O, SO₂, or (CR₄R₅)ₙ₃;
n₁ is 1, 2, or 3;
n₂ is for 2;
n₃ is 0, 1, 2, or 3;
wherein the ring represented by

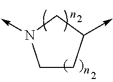

is optionally substituted by alkyl, halo, oxo, OR₆, NR₆R₇, or SR₆;
R₂ is selected from the following heteroaryl systems, optionally substituted:

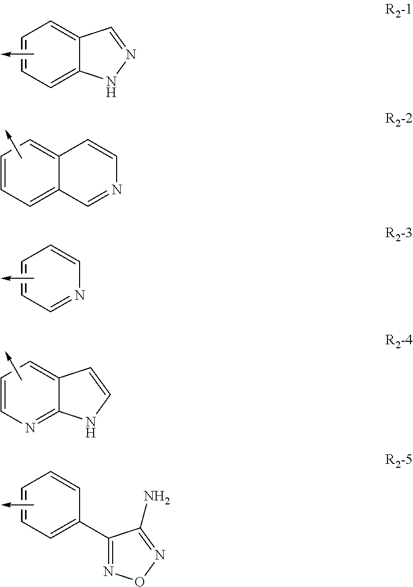

R₂-1 and R₂-1 are preferred;
R₃-R₇ are independently H, alkyl, alkenyl, alkynyl, cycloalkyl, cycloalkenyl, cycloalkylalkyl, cycloalkylalkenyl, or cycloalkylalkynyl optionally substituted.

Ar is a monocyclic or bicyclic aryl or heteroaryl ring, such as phenyl or naphthyl; X is from 1 to 3 substituents on Ar, each independently in the form Y—Z, in which Z is attached to Ar;

Y is one or more substituents on Z, and each is chosen independently from H, halogen, OR₈, NR₈R₉, NO₂, SR₈, SOR_{B}, SO₂R₈, SO₂NR₈R₉, NR₈SO₂R₉, OCF₃, CO₂R₈, CONR₈R₉, NR₈C(═O)R₉, NR₈C(═O)OR₉, OC(═O)NR₈R₉, NR₈C(═O)NR₉R₁₀, N-containing heterocycle, or N-containing heteroaryl (such as indazole and pyrazole);

Each instance of Z is chosen independently from alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, heterocycle, (heterocycle)alkyl, (heterocycle)alkenyl, (heterocycle)alkynyl, or is absent;

R₈-R₁₀ are independently absent, H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heterocycle, heteroaryl, heteroarylalkyl, heteroarylalkenyl, heteroarylalkynyl, (heterocycle)alkyl, (heterocycle)alkenyl, (heterocycle)alkynyl, or heterocycle; optionally substituted by one or more halogen or heteroatom-containing substituents, including but not limited to OR₁₁, COOR₁₁, NR₁₁R₁₂, NO₂, SR₁₁, SOR₁₁, SO₂R₁₁, SO₂NR₁₁R₁₂, NR₁₁SO₂R₁₂, OCF₃, CONR₁₁R₁₂, NR₁₁C(═O)R₁₂, NR₁₁C(═O)OR₁₂, OC(═O)NR₁₁R₁₂, or NR₁₁C(═O)NR₁₂R₁₃;

with any two of the groups R₈, R₉ and R₁₀ being optionally joined with a link selected from the group consisting of bond, —O—, —S—, —SO—, —SO₂—, and to form a ring; and

R₁₁-R₁₃ are independently H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, heterocycle, or are absent.

Preferred Z is alkyl or absent.

Preferred Q is (CR₄R₅)ₙ₃, and n₃ is 1-3. More preferred Q is CH₂.

Preferred R₃, R₄ and R₅ are H.

Preferred R₈-R₁₀ is H, alkyl, alkenyl, aryl, arylalkyl, arylalkenyl, cycloalkyl, cycloalkenyl, cycloalkylalkyl, cycloalkylalkenyl, heterocycle optionally substituted by OR₁₁, COOR₁₁, NR₁₁R₁₂, SO₂NR₁₁R₁₂, NR₁₁SO₂R₁₂ or absent; more preferred R₈ is H, alkyl, arylalkyl, cycloalkyl, cycloalkylalkyl, or heterocycle.

Preferred R₁₁-R₁₃ are H, alkyl, alkenyl, aryl, arylalkyl, arylalkenyl, cycloalkyl, cycloalkenyl, cycloalkylalkyl, cycloalkylalkenyl, or heterocycle.

A specific embodiment of Formula I is a compound of Formula III:

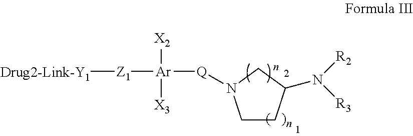

wherein X₂ and X₃ are the same as X and Z₁ is the same as Z, as defined above for Formula II. Y₁ is —O—, CO₂, —NR₈—, —SO₂NR₈— (N is connected to Z₁), —NR₈SO₂— (S is connected to Z₁), —NR₈CO— (C is connected to Z₁), or N-containing heteroaryl.

Preferred compounds of Formula II are shown in the following Table I. In the following structures, hydrogens are omitted from the drawings for the sake of simplicity. Tautomers drawn represent all tautomers possible. Structures are drawn to indicate the preferred stereochemistry; where stereoisomers may be generated in these compounds, structures are taken to mean any of the possible stereoisomers alone or a mixture of stereoisomers in any ratio.

TABLE I
Example Compounds of Formula II.

| Compound | Structure | FG1 |
|---|---|---|
| 1.091 | 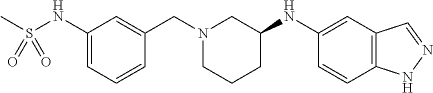 [SMILES: CS(=O)(=O)Nc1cccc(CN2CCC[C@H](Nc3ccc4[nH]ncc4c3)C2)c1] | sulfonamide |
| | (S)-N-(3-((3-(1H-indazol-5-ylamino)piperidin-1- | |
| | yl)methyl)phenyl)methanesulfonamide | |
| 1.093 | 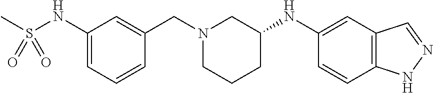 [SMILES: CS(=O)(=O)Nc1cccc(CN2CCC[C@@H](Nc3ccc4[nH]ncc4c3)C2)c1] | sulfonamide |
| | (R)-N-(3-((3-(1H-indazol-5-ylamino)piperidin-1- | |
| | yl)methyl)phenyl)methanesulfonamide | |
| 1.108 | 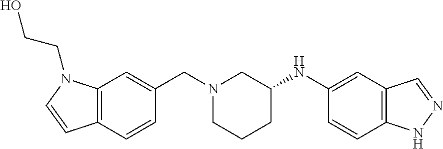 [SMILES: OCCn1ccc2ccc(CN3CCC[C@@H](Nc4ccc5[nH]ncc5c4)C3)cc21] | hydroxy |
| | (R)-2-(6-((3-(1H-indazol-5-ylamino)piperidin-1- | |
| | yl)methyl)-1H-indol-1-yl)ethanol | |
| 1.109 | 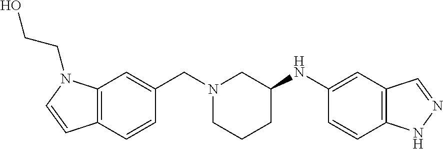 [SMILES: OCCn1ccc2ccc(CN3CCC[C@H](Nc4ccc5[nH]ncc5c4)C3)cc21] | hydroxy |
| | (S)-2-(6-((3-(1H-indazol-5-ylamino)piperidin-1- | |
| | yl)methyl)-1H-indol-1-yl)ethanol | |
| 1.123 | 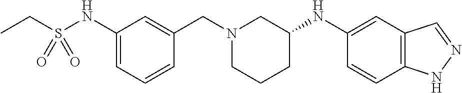 [SMILES: CCS(=O)(=O)Nc1cccc(CN2CCC[C@@H](Nc3ccc4[nH]ncc4c3)C2)c1] | sulfonamide |
| | (R)-N-(3-((3-(1H-indazol-5-ylamino)piperidin-1- | |
| | yl)methyl)phenyl)ethanesulfonamide | |
| 1.124 | 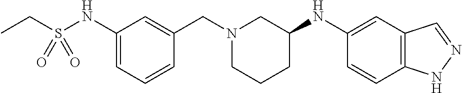 [SMILES: CCS(=O)(=O)Nc1cccc(CN2CCC[C@H](Nc3ccc4[nH]ncc4c3)C2)c1] | sulfonamide |
| | (S)-N-(3-((3-(1H-indazol-5-ylamino)piperidin-1- | |
| | yl)methyl)phenyl)ethanesulfonamide | |
| 1.148 | 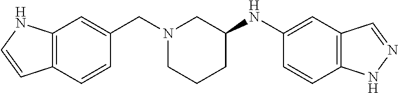 [SMILES: c1cc2ccc(CN3CCC[C@H](Nc4ccc5[nH]ncc5c4)C3)cc2[nH]1] | amine |
| | (S)-N-(1-((1H-indol-6-yl)methyl)piperidin-3-yl)-1H- | |
| | indazol-5-amine | |
| 1.149 | 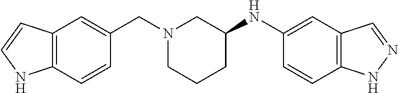 [SMILES: c1cc2cc(CN3CCC[C@H](Nc4ccc5[nH]ncc5c4)C3)ccc2[nH]1] | amine |
| | (S)-N-(1-((1H-indol-5-yl)methyl)piperidin-3-yl)-1H- | |
| | indazol-5-amine | |
| 1.152 | 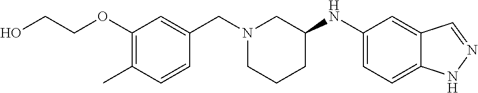 [SMILES: Cc1ccc(CN2CCC[C@H](Nc3ccc4[nH]ncc4c3)C2)cc1OCCO] | hydroxy |
| | (S)-2-(5-((3-(1H-indazol-5-ylamino)piperidin-1- | |
| | yl)methyl)-2-methylphenoxy)ethanol | |
| 1.161 | 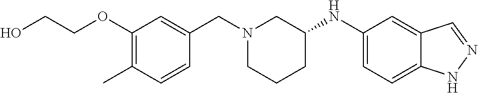 [SMILES: Cc1ccc(CN2CCC[C@@H](Nc3ccc4[nH]ncc4c3)C2)cc1OCCO] | hydroxy |
| | (R)-2-(5-((3-(1H-indazol-5-ylamino)piperidin-1- | |
| | yl)methyl)-2-methylphenoxy)ethanol | |
| 1.184 | 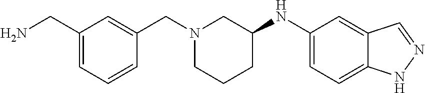 [SMILES: NCc1cccc(CN2CCC[C@H](Nc3ccc4[nH]ncc4c3)C2)c1] | amine |
| | (S)-N-(1-(3-(aminomethyl)benzyl)piperidin-3-yl)-1H- | |
| | indazol-5-amine | |
| 1.196 | 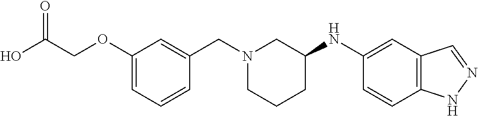 [SMILES: O=C(O)COc1cccc(CN2CCC[C@H](Nc3ccc4[nH]ncc4c3)C2)c1] | carboxylic acid |
| | (S)-2-(3-((3-(1H-indazol-5-ylamino)piperidin-1- | |
| | yl)methyl)phenoxy)acetic acid | |
| 1.212 | 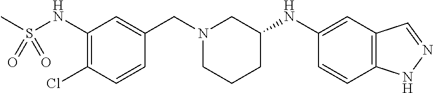 [SMILES: CS(=O)(=O)Nc1cc(CN2CCC[C@@H](Nc3ccc4[nH]ncc4c3)C2)ccc1Cl] | sulfonamide |
| | (R)-N-(5-((3-(1H-indazol-5-ylamino)piperidin-1- | |
| | yl)methyl)-2-chlorophenyl)methanesulfonamide | |
| 1.213 | 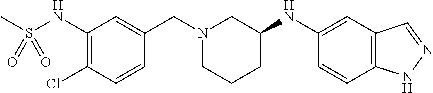 [SMILES: CS(=O)(=O)Nc1cc(CN2CCC[C@H](Nc3ccc4[nH]ncc4c3)C2)ccc1Cl] | sulfonamide |
| | (S)-N-(5-((3-(1H-indazol-5-ylamino)piperidin-1- | |
| | yl)methyl)-2-chlorophenyl)methanesulfonamide | |
| 1.217 | 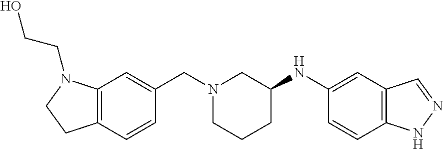 [SMILES: OCCN1CCc2ccc(CN3CCC[C@H](Nc4ccc5[nH]ncc5c4)C3)cc21] | hydroxy |
| | (S)-2-(6-((3-(1H-indazol-5-ylamino)piperidin-1- | |
| | yl)methyl)indolin-1-yl)ethanol | |
| 1.223 | 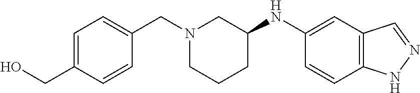 [SMILES: OCc1ccc(CN2CCC[C@H](Nc3ccc4[nH]ncc4c3)C2)cc1] | hydroxy |
| | (S)-(4-((3-(1H-indazol-5-ylamino)piperidin-1- | |
| | yl)methyl)phenyl)methanol | |
| 1.233 | 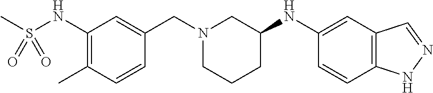 [SMILES: Cc1ccc(CN2CCC[C@H](Nc3ccc4[nH]ncc4c3)C2)cc1NS(C)(=O)=O] | sulfonamide |
| | (S)-N-(5-((3-(1H-indazol-5-ylamino)piperidin-1- | |
| | yl)methyl)-2-methylphenyl)methanesulfonamide | |
| 1.236 | 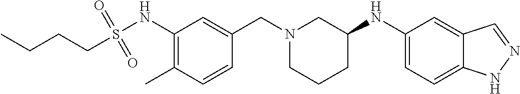 [SMILES: CCCCS(=O)(=O)Nc1cc(CN2CCC[C@H](Nc3ccc4[nH]ncc4c3)C2)ccc1C] | sulfonamide |
| | (S)-N-(5-((3-(1H-indazol-5-ylamino)piperidin-1- | |
| | yl)methyl)-2-methylphenyl)butane-1-sulfonamide | |
| 1.237 | 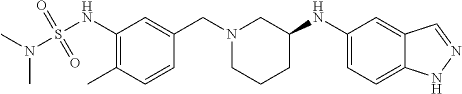 [SMILES: Cc1ccc(CN2CCC[C@H](Nc3ccc4[nH]ncc4c3)C2)cc1NS(=O)(=O)N(C)C] | sulfonamide |
| | (S)-N-(2-((3-(1H-indazol-5-ylamino)piperidin-1- | |
| | yl)methyl)-5-methylphenyl)-N′,N′- | |
| | dimethylaminosulfamide | |
| 1.238 | 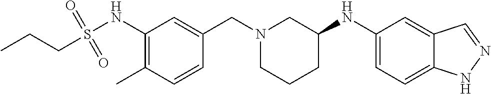 [SMILES: CCCS(=O)(=O)Nc1cc(CN2CCC[C@H](Nc3ccc4[nH]ncc4c3)C2)ccc1C] | sulfonamide |
| | (S)-N-(5-((3-(1H-indazol-5-ylamino)piperidin-1- | |
| | yl)methyl)-2-methylphenyl)propane-1-sulfonamide | |
| 1.239 | 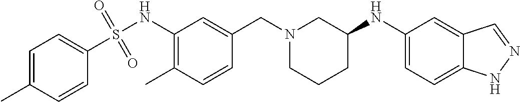 [SMILES: Cc1ccc(S(=O)(=O)Nc2cc(CN3CCC[C@H](Nc4ccc5[nH]ncc5c4)C3)ccc2C)cc1] | sulfonamide |
| | (S)-N-(5-((3-(1H-indazol-5-ylamino)piperidin-1- | |
| | yl)methyl)-2-methylphenyl)-4- | |
| | methylbenzenesulfonamide | |
| 1.252 | 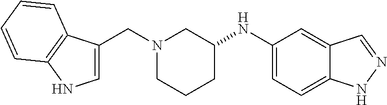 [SMILES: c1ccc2c(CN3CCC[C@@H](Nc4ccc5[nH]ncc5c4)C3)c[nH]c2c1] | amine |
| | (R)-N-(1-((1H-indol-3-yl)methyl)piperidin-3-yl)-1H- | |
| | indazol-5-amine | |
| 1.253 | 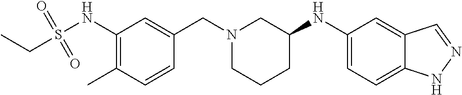 [SMILES: CCS(=O)(=O)Nc1cc(CN2CCC[C@H](Nc3ccc4[nH]ncc4c3)C2)ccc1C] | sulfonamide |
| | (S)-N-(5-((3-(1H-indazol-5-ylamino)piperidin-1- | |
| | yl)methyl)-2-methylphenyl)ethanesulfonamide | |
| 1.258 | 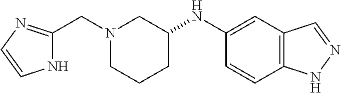 [SMILES: c1c[nH]c(CN2CCC[C@@H](Nc3ccc4[nH]ncc4c3)C2)n1] | amine |
| | (R)-N-(1-((1H-imidazol-2-yl)methyl)piperidin-3-yl)-1H- | |
| | indazol-5-amine | |
| 1.259 | 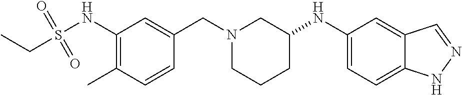 [SMILES: CCS(=O)(=O)Nc1cc(CN2CCC[C@@H](Nc3ccc4[nH]ncc4c3)C2)ccc1C] | sulfonamide |
| | (R)-N-(5-((3-(1H-indazol-5-ylamino)piperidin-1- | |
| | yl)methyl)-2-methylphenyl)ethanesulfonamide | |
| 1.260 | 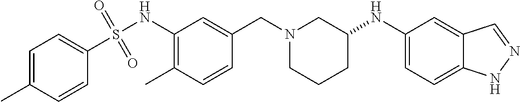 [SMILES: Cc1ccc(S(=O)(=O)Nc2cc(CN3CCC[C@@H](Nc4ccc5[nH]ncc5c4)C3)ccc2C)cc1] | sulfonamide |
| | (R)-N-(5-((3-(1H-indazol-5-ylamino)piperidin-1- | |
| | yl)methyl)-2-methylphenyl)-4- | |
| | methylbenzenesulfonamide | |
| 1.261 | 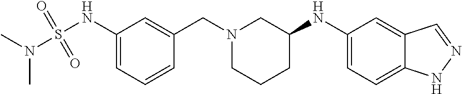 [SMILES: CN(C)S(=O)(=O)Nc1cccc(CN2CCC[C@H](Nc3ccc4[nH]ncc4c3)C2)c1] | sulfonamide |
| | (S)-N-(3-((3-(1H-indazol-5-ylamino)piperidin-1- | |
| | yl)methyl)phenyl)-N′,N′-dimethylaminosulfamide | |
| 1.262 | 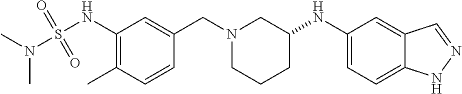 [SMILES: Cc1ccc(CN2CCC[C@@H](Nc3ccc4[nH]ncc4c3)C2)cc1NS(=O)(=O)N(C)C] | sulfonamide |
| | (R)-N-(2-((3-(1H-indazol-5-ylamino)piperidin-1- | |
| | yl)methyl)-5-methylphenyl)-N′,N′- | |
| | dimethylaminosulfamide | |
| 1.270 | 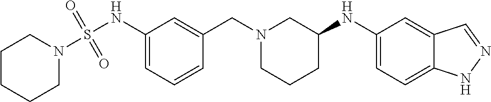 [SMILES: O=S(=O)(Nc1cccc(CN2CCC[C@H](Nc3ccc4[nH]ncc4c3)C2)c1)N1CCCCC1] | sulfonamide |
| | (S)-N-(3-((3-(1H-indazol-5-ylamino)piperidin-1- | |
| | yl)methyl)phenyl)piperidine-1-sulfonamide | |
| 1.273 | 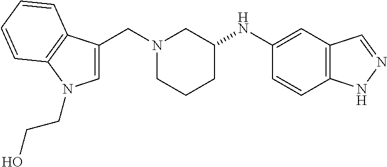 [SMILES: OCCn1cc(CN2CCC[C@@H](Nc3ccc4[nH]ncc4c3)C2)c2ccccc21] | hydroxy |
| | (R)-2-(3-((3-(1H-indazol-5-ylamino)piperidin-1- | |
| | yl)methyl)-1H-indol-1-yl)ethanol | |
| 1.275 | 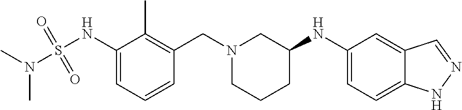 [SMILES: Cc1c(CN2CCC[C@H](Nc3ccc4[nH]ncc4c3)C2)cccc1NS(=O)(=O)N(C)C] | sulfonamide |
| | (S)-N-(3-((3-(1H-indazol-5-ylamino)piperidin-1- | |
| | yl)methyl)-2-methylphenyl)-N′,N′- | |
| | dimethylaminosulfamide | |
| 2.038 | 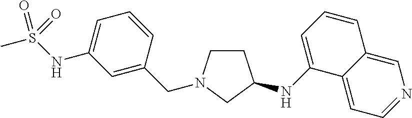 [SMILES: CS(=O)(=O)Nc1cccc(CN2CC[C@@H](Nc3cccc4cnccc34)C2)c1] | sulfonamide |
| | (R)-N-(3-((3-(isoquinolin-5-ylamino)pyrrolidin-1- | |
| | yl)methyl)phenyl)methanesulfonamide | |
| 2.039 | 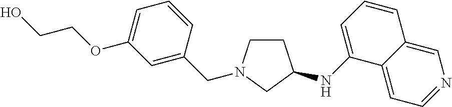 [SMILES: OCCOc1cccc(CN2CC[C@@H](Nc3cccc4cnccc34)C2)c1] | hydroxy |
| | (R)-2-(3-((3-(isoquinolin-5-ylamino)pyrrolidin-1- | |
| | yl)methyl)phenoxy)ethanol | |
| 2.041 | 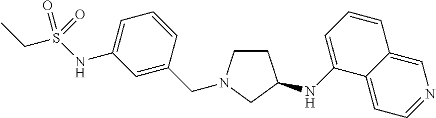 [SMILES: CCS(=O)(=O)Nc1cccc(CN2CC[C@@H](Nc3cccc4cnccc34)C2)c1] | sulfonamide |
| | (R)-N-(3-((3-(isoquinolin-5-ylamino)pyrrolidin-1- | |
| | yl)methyl)phenyl)ethanesulfonamide | |
| 2.054 | 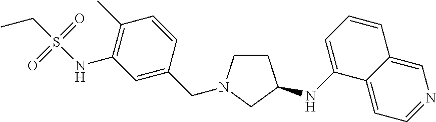 [SMILES: CCS(=O)(=O)Nc1cc(CN2CC[C@@H](Nc3cccc4cnccc34)C2)ccc1C] | sulfonamide |
| | (R)-N-(5-((3-(isoquinolin-5-ylamino)pyrrolidin-1- | |
| | yl)methyl)-2-methylphenyl)ethanesulfonamide | |
| 2.057 | 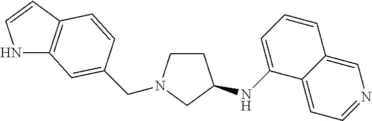 [SMILES: c1cc(N[C@@H]2CCN(Cc3ccc4cc[nH]c4c3)C2)c2ccncc2c1] | amine |
| | (R)-N-(1-((1H-indol-6-yl)methyl)pyrrolidin-3- | |
| | yl)isoquinolin-5-amine | |
| 2.060 | 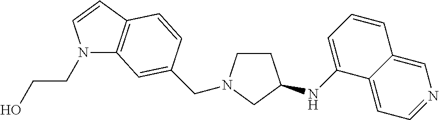 [SMILES: OCCn1ccc2ccc(CN3CC[C@@H](Nc4cccc5cnccc45)C3)cc21] | hydroxy |
| | (R)-2-(6-((3-(isoquinolin-5-ylamino)pyrrolidin-1- | |
| | yl)methyl)-1H-indol-1-yl)ethanol | |
| 2.064 | 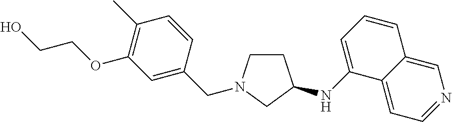 [SMILES: Cc1ccc(CN2CC[C@@H](Nc3cccc4cnccc34)C2)cc1OCCO] | hydroxy |
| | (R)-2-(5-((3-(isoquinolin-5-ylamino)pyrrolidin-1- | |
| | yl)methyl)-2-methylphenoxy)ethanol | |
| 2.065 | 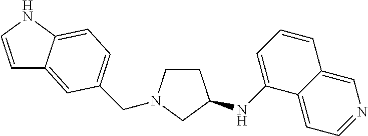 [SMILES: c1cc(N[C@@H]2CCN(Cc3ccc4[nH]ccc4c3)C2)c2ccncc2c1] | amine |
| | (R)-N-(1-((1H-indol-5-yl)methyl)pyrrolidin-3- | |
| | yl)isoquinolin-5-amine | |
| 2.066 | 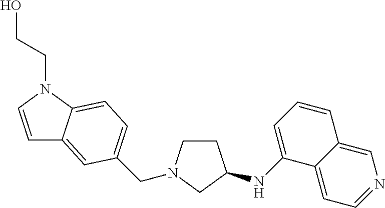 [SMILES: OCCn1ccc2cc(CN3CC[C@@H](Nc4cccc5cnccc45)C3)ccc21] | hydroxy |
| | (R)-2-(5-((3-(isoquinolin-5-ylamino)pyrrolidin-1- | |
| | yl)methyl)-1H-indol-1-yl)ethanol | |
| 2.067 | 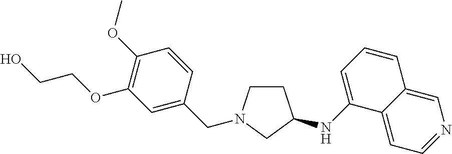 [SMILES: COc1ccc(CN2CC[C@@H](Nc3cccc4cnccc34)C2)cc1OCCO] | hydroxy |
| | (R)-2-(5-((3-(isoquinolin-5-ylamino)pyrrolidin-1- | |
| | yl)methyl)-2-methoxyphenoxy)ethanol | |
| 2.068 | 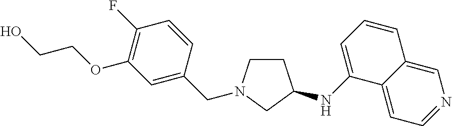 [SMILES: OCCOc1cc(CN2CC[C@@H](Nc3cccc4cnccc34)C2)ccc1F] | hydroxy |
| | (R)-2-(2-fluoro-5-((3-(isoquinolin-5-ylamino)pyrrolidin- | |
| | 1-yl)methyl)phenoxy)ethanol | |
| 2.069 | 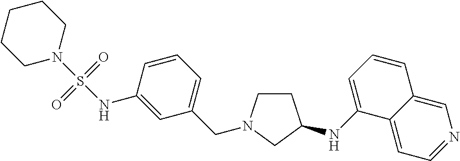 [SMILES: O=S(=O)(Nc1cccc(CN2CC[C@@H](Nc3cccc4cnccc34)C2)c1)N1CCCCC1] | sulfonamide |
| | (R)-N-(3-((3-(isoquinolin-5-ylamino)pyrrolidin-1- | |
| | yl)methyl)phenyl)piperidine-1-sulfonamide | |
| 2.072 | 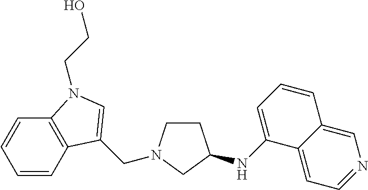 [SMILES: OCCn1cc(CN2CC[C@@H](Nc3cccc4cnccc34)C2)c2ccccc21] | hydroxy |
| | (R)-2-(3-((3-(isoquinolin-5-ylamino)pyrrolidin-1- | |
| | yl)methyl)-1H-indol-1-yl)ethanol | |
| 2.073 | 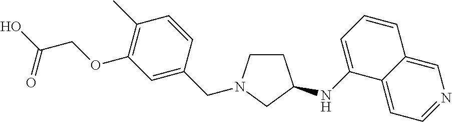 [SMILES: Cc1ccc(CN2CC[C@@H](Nc3cccc4cnccc34)C2)cc1OCC(=O)O] | carboxylic acid |
| | (R)-2-(5-((3-(isoquinolin-5-ylamino)pyrrolidin-1- | |
| | yl)methyl)-2-methylphenoxy)acetic acid | |
| 2.076 | 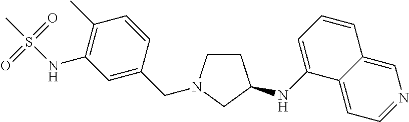 [SMILES: Cc1ccc(CN2CC[C@@H](Nc3cccc4cnccc34)C2)cc1NS(C)(=O)=O] | sulfonamide |
| | (R)-N-(5-((3-(isoquinolin-5-ylamino)pyrrolidin-1- | |
| | yl)methyl)-2-methylphenyl)methanesulfonamide | |
| 2.077 | 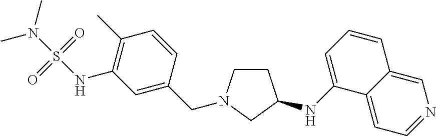 [SMILES: Cc1ccc(CN2CC[C@@H](Nc3cccc4cnccc34)C2)cc1NS(=O)(=O)N(C)C] | sulfonamide |
| | (R)-N-(5-((3-(isoquinolin-5-ylamino)pyrrolidin-1- | |
| | yl)methyl)-2-methylphenyl)-N′,N′- | |
| | dimethylaminosulfamide | |
| 2.078 | 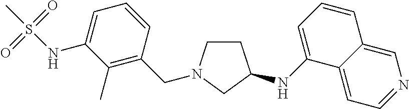 [SMILES: Cc1c(CN2CC[C@@H](Nc3cccc4cnccc34)C2)cccc1NS(C)(=O)=O] | sulfonamide |
| | (R)-N-(3-((3-(isoquinolin-5-ylamino)pyrrolidin-1- | |
| | yl)methyl)-2-methylphenyl)methanesulfonamide | |
| 2.079 | 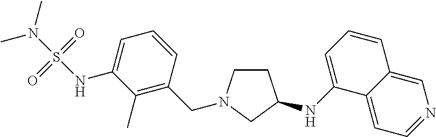 [SMILES: Cc1c(CN2CC[C@@H](Nc3cccc4cnccc34)C2)cccc1NS(=O)(=O)N(C)C] | sulfonamide |
| | (R)-N-(3-((3-(isoquinolin-5-ylamino)pyrrolidin-1- | |
| | yl)methyl)-2-methylphenyl)-N′,N′- | |
| | dimethylaminosulfamide | |
| 6.001 | 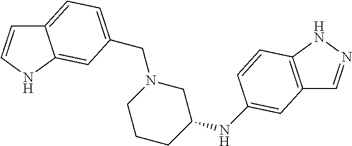 [SMILES: c1cc2ccc(CN3CCC[C@@H](Nc4ccc5[nH]ncc5c4)C3)cc2[nH]1] | amine |
| | (R)-N-(1-((1H-indol-6-yl)methyl)piperidin-3-yl)-1-H- | |
| | indazol-5-amine | |
| 6.002 | 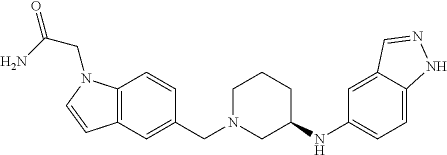 [SMILES: NC(=O)Cn1ccc2cc(CN3CCC[C@@H](Nc4ccc5[nH]ncc5c4)C3)ccc21] | amine |
| | (R)-2-(5-((3-(1H-indazol-5-ylamino)piperidin-1- | |
| | yl)methyl)-1H-indol-1-yl)acetamide | |
| 6.003 | 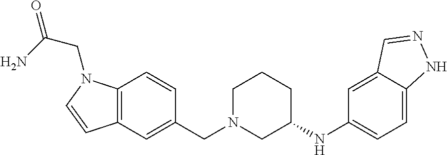 [SMILES: NC(=O)Cn1ccc2cc(CN3CCC[C@H](Nc4ccc5[nH]ncc5c4)C3)ccc21] | amine |
| | (S)-2-(5-((3-(1H-indazol-5-ylamino)piperidin-1- | |
| | yl)methyl)-1H-indol-1-yl)acetamide | |
| 6.004 | 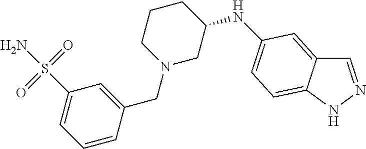 [SMILES: NS(=O)(=O)c1cccc(CN2CCC[C@H](Nc3ccc4[nH]ncc4c3)C2)c1] | sulfonamide |
| | (S)-3-((3-(1H-indazol-5-ylamino)piperidin-1- | |
| | yl)methyl)benzenesulfonamide | |
| 6.005 | 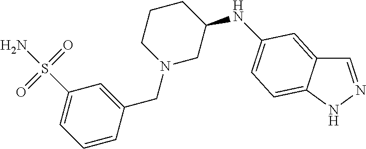 [SMILES: NS(=O)(=O)c1cccc(CN2CCC[C@@H](Nc3ccc4[nH]ncc4c3)C2)c1] | sulfonamide |
| | (R)-3-((3-(1H-indazol-5-ylamino)piperidin- | |
| | yl)methyl)benzenesulfonamide | |
| 6.006 | 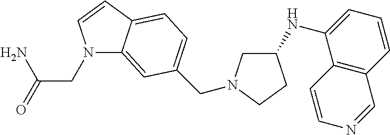 [SMILES: NC(=O)Cn1ccc2ccc(CN3CC[C@@H](Nc4cccc5cnccc45)C3)cc21] | amine |
| | (R)-2-(6-((3-(isoquinolin-5-ylamino)pyrrolidin-1- | |
| | yl)methyl)-1H-indol-1-yl)acetamide | |
| 6.007 | 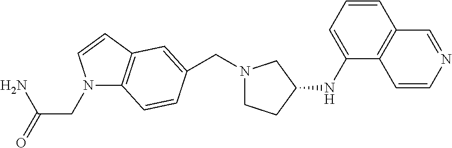 [SMILES: NC(=O)Cn1ccc2cc(CN3CC[C@@H](Nc4cccc5cnccc45)C3)ccc21] | amine |
| | (R)-2-(5-((3-(isoquinolin-5-ylamino)pyrrolidin-1- | |
| | yl)methyl)-1H-indol-1-yl)acetamide | |

The compounds of this invention are particularly directed towards Formula I and III compounds in which Drug₂ is selected from derivatives of the muscarinic agonist pilocarpine (Drug₂-1), or the prostaglandin F_{2α} agonists (Drug₂-2), and FG₂ is a carboxylic acid, which is connected to Link through COO—.

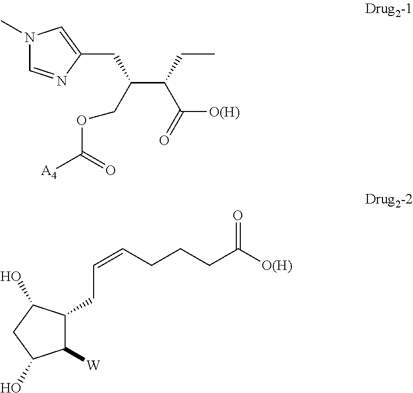

Preferred groups W of Drug₂-2 are W-1, W-2, W-3, W-4, and W-5, shown below.

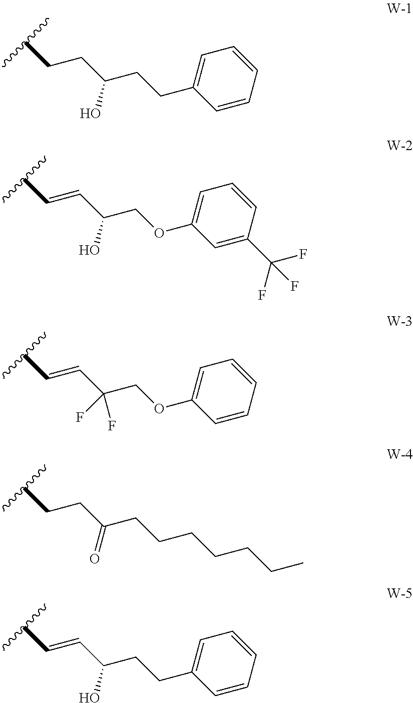

In these compounds, A₄ is alkyl, cycloalkyl, cycloalkylalkyl, or arylalkyl, and W represents the functionality well known in the literature of prostaglandin F_{2α} receptor agonists.

Pilocarpine (Drug₂-1) is a muscarinic alkaloid obtained from the leaves of tropical American shrubs, from the genus Pilocarpus. It is the most widely used cholinergic drug for the treatment of glaucoma. It acts by stimulating the muscarinic receptors of the ciliary muscle, which widens the anterior chamber angle, resulting in an increased outflow of aqueous humor through the trabecular meshwork.

Prostaglandins (Drug₂-2) are known mediators of inflammation and at low doses; prostaglandins have been shown to lower TOP. Hypotensive lipids, known as eicosanoids, include the prostaglandin analogs latanoprost, travaprost and bimatoprost. As an example, latanoprost, which is an ester prodrug analogue of a prostaglandin F_{2α} analogue, is a selective prostanoid FP receptor agonist. Latanoprost reduces TOP by increasing the aqueous outflow from the eye, through the uveoscleral pathway. How this occurs is not known, but it is thought that they bind to the receptors of the ciliary body and upregulate metalloproteinases. These enzymes remodel the extracellular matrix and make the area more permeable to aqueous humor, thereby increasing outflow.

TABLE 2
Exemplified Compounds of Formula III.

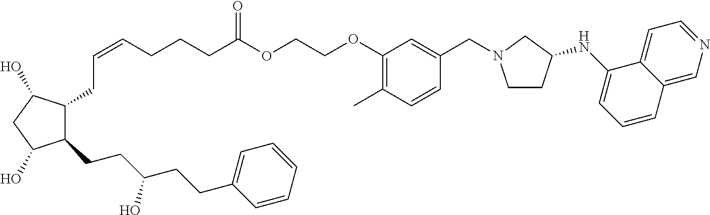 [SMILES: Cc1ccc(CN2CC[C@@H](Nc3cccc4cnccc34)C2)cc1OCCOC(=O)CCC/C=C\C[C@@H]1[C@@H](CC[C@@H](O)CCc2ccccc2)[C@H](O)C[C@@H]1O]

(Z)-2-(5-(((R)-3-(isoquinolin-5-ylamino)pyrrolidin-1-yl)methyl)-2-methylphenoxy)ethyl
7-((1R,2R,3R,5S)-3,5-dihydroxy-2-((R)-3-hydroxy-5-phenylpentyl)cyclopentyl)hept-5-
enoate, Compound 1

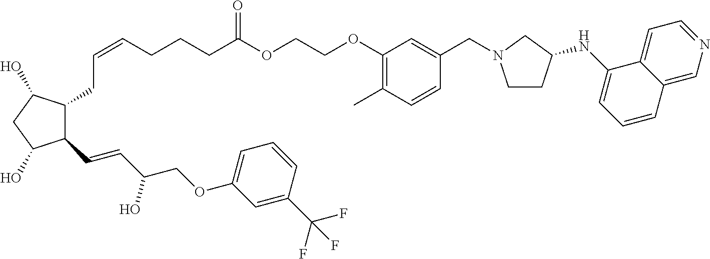 [SMILES: Cc1ccc(CN2CC[C@@H](Nc3cccc4cnccc34)C2)cc1OCCOC(=O)CCC/C=C\C[C@@H]1[C@@H](/C=C/[C@@H](O)COc2cccc(C(F)(F)F)c2)[C@H](O)C[C@@H]1O]

(Z)-2-(5-(((R)-3-(isoquinolin-5-ylamino)pyrrolidin-1-yl)methyl)-2-methylphenoxy)ethyl
7-((1R,2R,3R,5S)-3,5-dihydroxy-2-((R,E)-3-hydroxy-4-(3-(trifluoromethyl)phenoxy)but-
1-enyl)cyclopentyl)hept-5-enoate, Compound 2

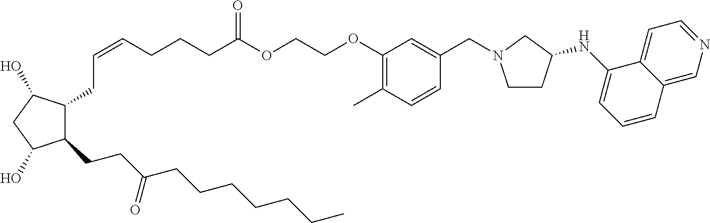 [SMILES: CCCCCCCC(=O)CC[C@@H]1[C@@H](C/C=C\CCCC(=O)OCCOc2cc(CN3CC[C@@H](Nc4cccc5cnccc45)C3)ccc2C)[C@@H](O)C[C@H]1O]

(Z)-2-(5-(((R)-3-(isoquinolin-5-ylamino)pyrrolidin-1-yl)methyl)-2-methylphenoxy)ethyl
7-((1R,2R,3R,5S)-3,5-dihydroxy-2-(3-oxodecyl)cyclopentyl)hept-5-enoate, Compound 3

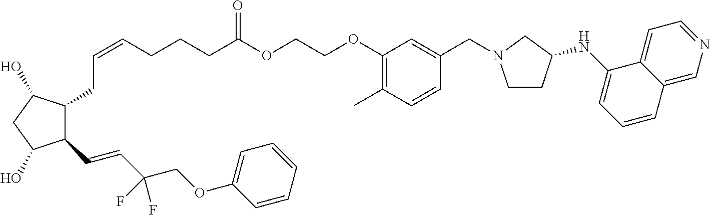 [SMILES: Cc1ccc(CN2CC[C@@H](Nc3cccc4cnccc34)C2)cc1OCCOC(=O)CCC/C=C\C[C@@H]1[C@@H](/C=C/C(F)(F)COc2ccccc2)[C@H](O)C[C@@H]1O]

(Z)-2-(5-(((R)-3-(isoquinolin-5-ylamino)pyrrolidin-1-yl)methyl)-2-methylphenoxy)ethyl
7-((1R,2R,3R,5S)-2-((E)-3,3-difluoro-4-phenoxybut-1-enyl)-3,5-
dihydroxycyclopentyl)hept-5-enoate, Compound 4

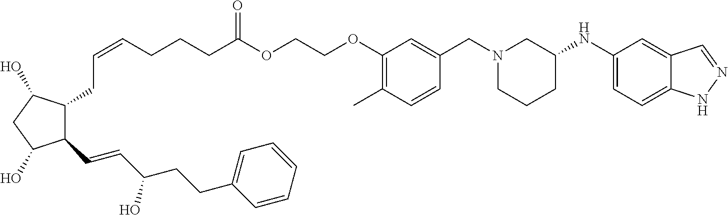 [SMILES: Cc1ccc(CN2CCC[C@@H](Nc3ccc4[nH]ncc4c3)C2)cc1OCCOC(=O)CCC/C=C\C[C@@H]1[C@@H](/C=C/[C@@H](O)CCc2ccccc2)[C@H](O)C[C@@H]1O]

(5Z)-2-(5-(((R)-3-(1H-indazol-5-ylamino)piperidin-1-yl)methyl)-2-methylphenoxy)ethyl
7-((1R,2R,3R,5S)-3,5-dihydroxy-2-((S,E)-3-hydroxy-5-phenylpent-1-
enyl)cyclopentyl)hept-5-enoate, Compound 5

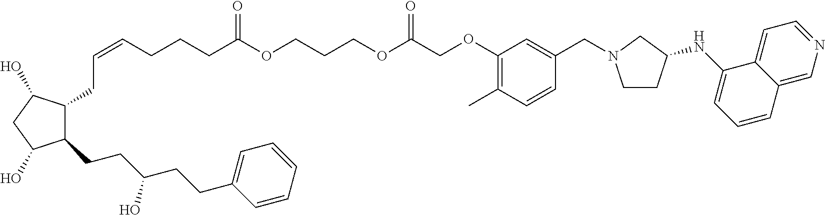 [SMILES: Cc1ccc(CN2CC[C@@H](Nc3cccc4cnccc34)C2)cc1OCC(=O)OCCCOC(=O)CCC/C=C\C[C@@H]1[C@@H](CC[C@@H](O)CCc2ccccc2)[C@H](O)C[C@@H]1O]

(Z)-3-(2-(5-(((R)-3-(isoquinolin-5-ylamino)pyrrolidin-1-yl)methyl)-2-
methylphenoxy)propyl 7-((1R,2R,3R,5S)-3,5-dihydroxy-2-((R)-3-hydroxy-5-
phenylpentyl)cyclopentyl)hept-5-enoate, Compound 6

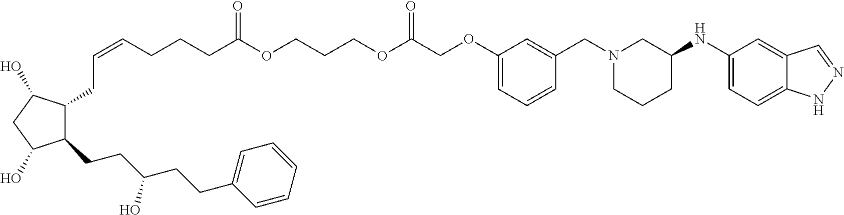 [SMILES: O=C(CCC/C=C\C[C@@H]1[C@@H](CC[C@@H](O)CCc2ccccc2)[C@H](O)C[C@@H]1O)OCCCOC(=O)COc1cccc(CN2CCC[C@H](Nc3ccc4[nH]ncc4c3)C2)c1]

(Z)-3-(2-(3-(((S)-3-(1H-indazol-5-ylamino)piperidin-1-
yl)methyl)phenoxy)acetoxy)propyl 7-((1R,2R,3R,5S)-3,5-dihydroxy-2-((R)-3-hydroxy-5-
phenylpentyl)cyclopentyl)hept-5-enoate, Compound 7

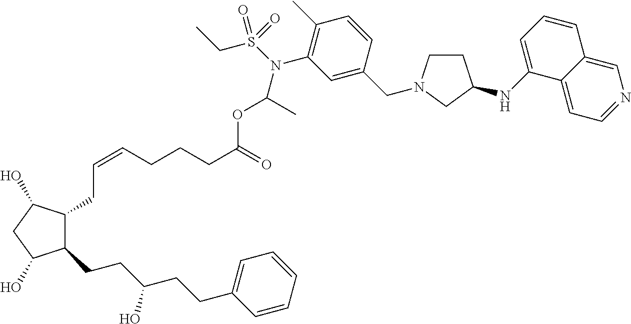 [SMILES: CCS(=O)(=O)N(c1cc(CN2CC[C@@H](Nc3cccc4cnccc34)C2)ccc1C)C(C)OC(=O)CCC/C=C\C[C@@H]1[C@@H](CC[C@@H](O)CCc2ccccc2)[C@H](O)C[C@@H]1O]

(Z)-1-(N-(5-(((R)-3-(isoquinolin-5-ylamino)pyrrolidin-1-yl)methyl)-2-
methylphenyl)ethylsulfonamido)ethyl 7-((1R,2R,3R,5S)-3,5-dihydroxy-2-((R)-3-hydroxy-
5-phenylpentyl)cyclopentyl)hept-5-enoate, Compound 8

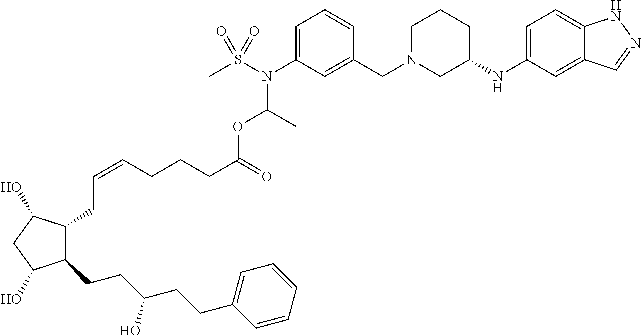 [SMILES: CC(OC(=O)CCC/C=C\C[C@@H]1[C@@H](CC[C@@H](O)CCc2ccccc2)[C@H](O)C[C@@H]1O)N(c1cccc(CN2CCC[C@H](Nc3ccc4[nH]ncc4c3)C2)c1)S(C)(=O)=O]

(Z)-1-(N-(3-(((S)-3-(1H-indazol-5-ylamino)piperidin-1-
yl)methyl)phenyl)methylsulfonamido)ethyl 7-((1R,2R,3R,5S)-3,5-dihydroxy-2-((R)-3-
hydroxy-5-phenylpentyl)cyclopentyl)hept-5-enoate, Compound 9

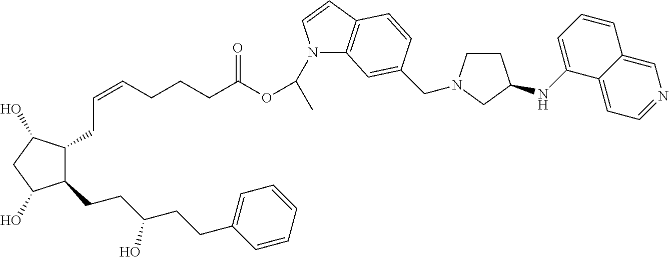 [SMILES: CC(OC(=O)CCC/C=C\C[C@@H]1[C@@H](CC[C@@H](O)CCc2ccccc2)[C@H](O)C[C@@H]1O)n1ccc2ccc(CN3CC[C@@H](Nc4cccc5cnccc45)C3)cc21]

(Z)-1-(6-(((R)-3-(isoquinolin-5-ylamino)pyrrolidin-1-yl)methyl)-1H-indol-1-yl)ethyl 7-
((1R,2R,3R,5S)-3,5-dihydroxy-2-((R)-3-hydroxy-5-phenylpentyl)cyclopentyl)hept-5-
enoate, Compound 10

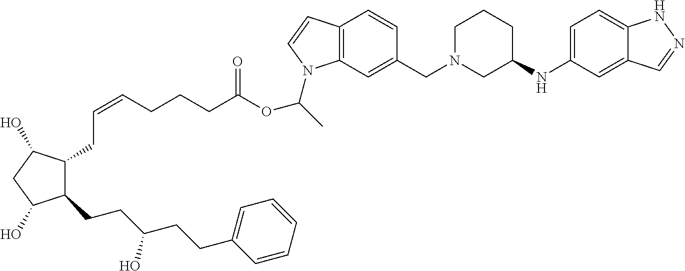 [SMILES: CC(OC(=O)CCC/C=C\C[C@@H]1[C@@H](CC[C@@H](O)CCc2ccccc2)[C@H](O)C[C@@H]1O)n1ccc2ccc(CN3CCC[C@@H](Nc4ccc5[nH]ncc5c4)C3)cc21]

(Z)-1-(6-(((R)-3-(1H-indazol-5-ylamino)piperidin-1-yl)methyl)-1H-indol-1-yl)ethyl 7-
((1R,2R,3R,5S)-3,5-dihydroxy-2-((R)-3-hydroxy-5-phenylpentyl)cyclopentyl)hept-5-
enoate, Compound 11

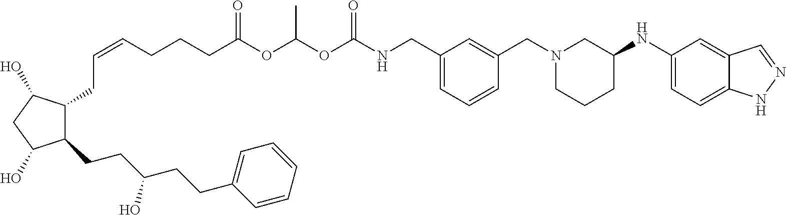 [SMILES: CC(OC(=O)CCC/C=C\C[C@@H]1[C@@H](CC[C@@H](O)CCc2ccccc2)[C@H](O)C[C@@H]1O)OC(=O)NCc1cccc(CN2CCC[C@H](Nc3ccc4[nH]ncc4c3)C2)c1]

(Z)-1-(3-(((S)-3-(1H-indazol-5-ylamino)piperidin-1-yl)methyl)benzylcarbamoyloxy)ethyl
7-((1R,2R,3R,5S)-3,5-dihydroxy-2-((R)-3-hydroxy-5-phenylpentyl)cyclopentyl)hept-5-
enoate, Compound 12

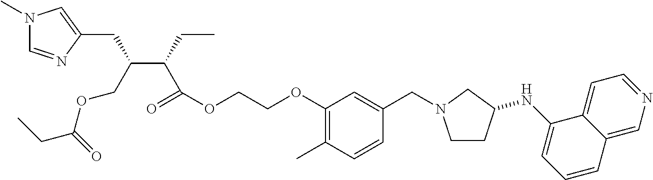 [SMILES: CCC(=O)OC[C@H](Cc1cn(C)cn1)[C@H](CC)C(=O)OCCOc1cc(CN2CC[C@@H](Nc3cccc4cnccc34)C2)ccc1C]

(2S,3R)-2-(5-(((R)-3-(isoquinolin-5-ylamino)pyrrolidin-1-yl)methyl)-2-
methylphenoxy)ethyl 2-ethyl-4-(1-methyl-1H-imidazol-4-yl)-3-
(propionyloxymethyl)butanoate, Compound 13

### Preparation of Compounds of Formula I and Formula III

The present invention is additionally directed to procedures for preparing compounds of Formula I. General approaches for preparations of the compounds of the Formula, particularly those compounds described by Formula III, are described in Scheme 1. Those having skill in the art will recognize that the starting materials can be varied and additional steps can be employed to produce compounds encompassed by the present invention. In some cases, protection of certain reactive functionalities may be necessary to achieve some of the above transformations. In general, the need for such protecting groups as well as the conditions necessary to attach and remove such groups will be apparent to those skilled in the art of organic synthesis.

Those skilled in the art will recognize various synthetic methodologies that can be employed to prepare non-toxic pharmaceutically acceptable prodrugs, for example acylated prodrugs, of the compounds of this invention.

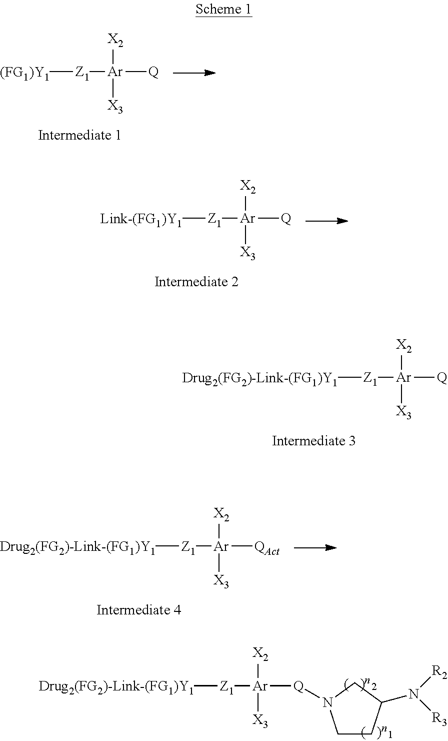

Compounds of the general form of Intermediate 1 are well described in the literature. Reaction of these intermediates with an appropriately activated form of the linking functionality provides compounds of the form Intermediate 2. These intermediate compounds may then be reacted with Drug₂(FG₂) to afford the intermediate compounds Intermediate 3. In some cases, further reaction of Intermediate 3 will require activation of the functionality in Q to provide the active form in Intermediate 4. This intermediate may then be coupled with the remainder of the molecule to provide the compounds of Formula III.

Those skilled in the art will recognize that some synthetic operations will benefit from protection of the functionality in Drug₂-2, as shown below, and the nature and choice of the appropriate protecting group (PG) will be clear.

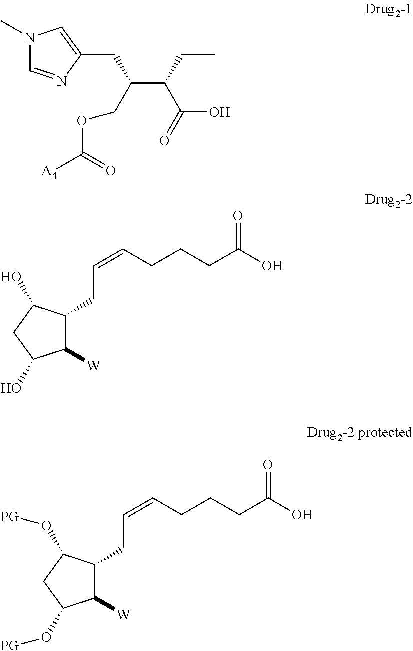

In one specific embodiment, the ROCK inhibitor portion Drug₁ bears a hydroxyl group for Fth. In these cases, linking groups of the form Link-1 and Link-2 are preferred. The general methods for preparing compounds of this type are shown in Scheme 2.

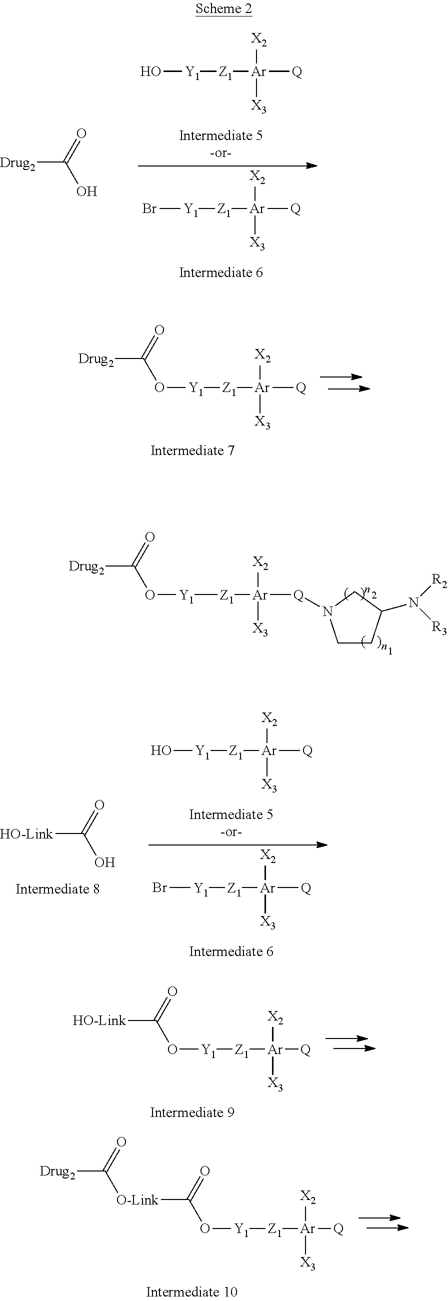

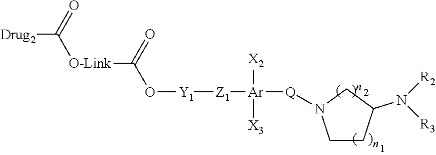

In the case of Link-1, in which the linking group is absent, direct coupling of the alcohol Intermediate 5 with the Drug₂ carboxylate yields the coupled product Intermediate 7. Methods for accomplishing this coupling are well know to those skilled in the art, and include direct esterification, esterification mediated by coupling agents such as carbodiimides, or activation of the carboxylic acid, for example as the acid halide, and subsequent coupling. Alternatively, the alcohol partner can be activated, for example using the Mitsunobu reaction, by conversion to a halide such as the bromide shown in Intermediate 6, or other activated forms such as a mesylate or tosylate, and these activated intermediates displaced by the carboxylate, typically in the presence of base catalysis. Some of these methods will result in the inversion of the stereochemical configuration at the alcohol center, if this is a chiral center in the molecule. Those skilled in the art will recognize the occurrence of these situations and how to adjust the chemistry to obtain the desired products. Further elaboration of Intermediate 7 as described for Scheme I provides the desired compound of Formula III.

In a variation of the above preparation, the linking group Link-2 can be incorporated by coupling the link unit in the form of Intermediate 8 with either Intermediate 5 or Intermediate 6, as described above, to provide Intermediate 9. This intermediate can then be coupled to the Drug₂ carboxylate as previously described to afford intermediate 10, which is converted in a fashion analogous to that described for Intermediate 7 to the desired compound of Formula III in which the linking group is Link-2.

In another specific embodiment of the invention, the ROCK inhibitor portion Drug₁ bears a carboxylic acid group for FG₁. In these cases, linking groups of the form Link-3 are preferred. The general methods for preparing compounds of this type are shown in Scheme 3.

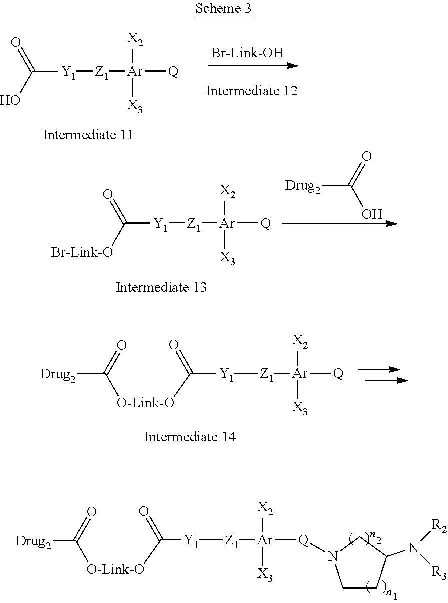

In this case, Intermediate 11, which bears the carboxylic acid FG₁ of Drug₁, is esterified with Intermediate 12 to yield Intermediate 13. Any of the esterification methods described above for Link-1 in Scheme II can be used for this transformation. Subsequently, reaction of the Drug₂ carboxylate with Intermediate 13 in a nucleophilic displacement of the halide provides the diester Intermediate 14. Further elaboration of Intermediate 14 as described for Scheme I provides the desired compound of Formula III. It will be recognized that many variations are possible in the order of steps and in the nature of the coupling methods used to prepare the diester in Intermediate 14, several of which have been described above for the cases in Scheme II, and that these methods are all useful in the preparation of intermediates of this type.

In another specific embodiment of the invention, the ROCK inhibitor portion Drug₁ contains a nitrogen bearing a relatively acidic hydrogen as FG₁. Examples of such functionality include sulfonamide nitrogen atoms, particularly aryl amine sulfonamides, and the nitrogen atoms of many nitrogen-containing heterocyclic systems, such as indole or benzimidazole. In these cases, linking groups of the form Link-3 are preferred. The general methods for preparing compounds of this type are shown in Scheme 4.

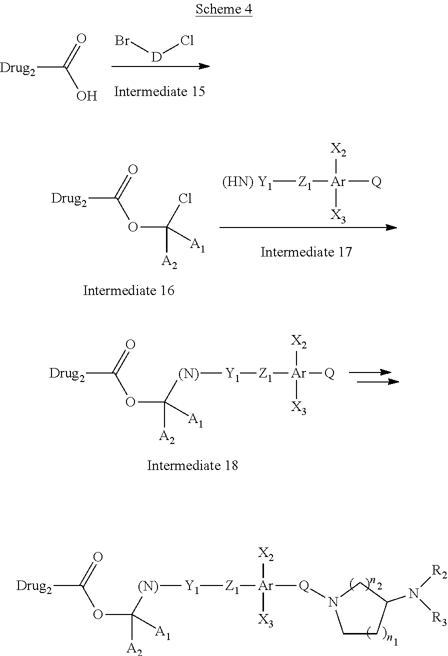

In this case, reaction of the Drug₂ carboxylate with a dihalocarbon such as Intermediate 15, typically under base catalysis, provides the haloalkyl ester Intermediate 16. Base catalyzed nucleophilic displacement of the halogen by the nitrogen in Intermediate 18, in which the relatively acidic hydrogen bearing nitrogen of FG₁ is represented schematically as (HN), provides the coupled Intermediate 18. Further elaboration of Intermediate 18 as described for Scheme I provides the desired compound of Formula III. The bromochlorocarbon Intermediate 15 is used here by example, and it will be recognized that many useful alternatives exist for the preparation of Intermediate 16.

In another specific embodiment of the invention, the ROCK inhibitor portion Drug₁ bears a functional group containing a nucleophilic nitrogen, such as a primary or secondary amine, for FG₁. In these cases, linking groups of the form Link-4 are preferred. The general methods for preparing compounds of this type are shown in Scheme 5.

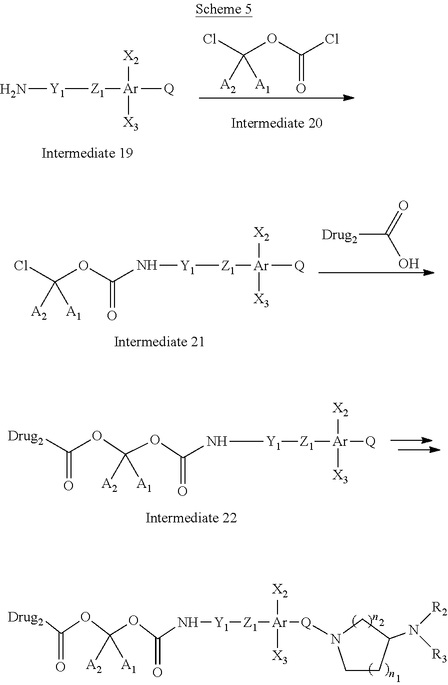

In the case of Link-4, Intermediate 19, which bears the nucelophilic nitrogen FG₁ of Drug₁, shown here as a primary amine for the purpose of exemplification, is acylated with the haloalkyl chloroformate Intermediate 20, typically in the presence of base, to afford the carbamate product Intermediate 21. Nucleophilic displacement of the halogen in Intermediate 21 by the carboxylate of Drug₂, also typically in the presence of base, provides the ester acetal carbamate Intermediate 22. Further elaboration of Intermediate 22 as described for Scheme I provides the desired compound of Formula III.

### Pharmaceutical Composition and Use

The present invention provides pharmaceutical compositions comprising a pharmaceutically acceptable carrier and one or more compounds of Formula III, pharmaceutically acceptable salts, solvates, and/or hydrates thereof. The pharmaceutically acceptable carrier can be selected by those skilled in the art using conventional criteria. Pharmaceutically acceptable carriers include, but are not limited to, aqueous- and non-aqueous based solutions, suspensions, emulsions, microemulsions, micellar solutions, gels, and ointments. The pharmaceutically active carriers may also contain ingredients that include, but are not limited to, saline and aqueous electrolyte solutions; ionic and nonionic osmotic agents such as sodium chloride, potassium chloride, glycerol, and dextrose; pH adjusters and buffers such as salts of hydroxide, hydronium, phosphate, citrate, acetate, borate, and tromethamine; antioxidants such as salts, acids and/or bases of bisulfite, sulfite, metabisulfite, thiosulfite, ascorbic acid, acetyl cysteine, cystein, glutathione, butylated hydroxyanisole, butylated hydroxytoluene, tocopherols, and ascorbyl palmitate; surfactants such as phospholipids (e.g., phosphatidylcholine, phosphatidylethanolamine and phosphatidyl inositiol), poloxamers and ploxamines, polysorbates such as polysorbate 80, polysorbate 60, and polysorbate 20, polyethers such as polyethylene glycols and polypropylene glycols; polyvinyls such as polyvinyl alcohol and povidone; cellulose derivatives such as methylcellulose, hydroxypropyl cellulose, hydroxyethyl cellulose, carboxymethyl cellulose and hydroxypropyl methylcellulose and their salts; petroleum derivatives such as mineral oil and white petrolatum; fats such as lanolin, peanut oil, palm oil, soybean oil; mono-, di-, and triglycerides; polymers of acrylic acid such as carboxypolymethylene gel, and polysaccharides such as dextrans, and glycosaminoglycans such as sodium hyaluronate. Such pharmaceutically acceptable carriers may be preserved against bacterial contamination using well-known preservatives, these include, but are not limited to, benzalkonium chloride, ethylene diamine tetra-acetic acid and its salts, benzethonium chloride, chlorhexidine, chlorobutanol, methylparaben, thimerosal, and phenylethyl alcohol, or may be formulated as a non-preserved formulation for either single or multiple use.

In one embodiment of the invention, the compositions are formulated as topical ophthalmic preparations, with a pH of about 3-9, preferably 4 to 8. The compounds of the invention are generally contained in these formulations in an amount of at least 0.001% by weight, for example, 0.001% to 5% by weight, preferably about 0.003% to about 2% by weight, with an amount of about 0.02% to about 1% by weight being most preferred. For topical administration, one to two drops of these formulations are delivered to the surface of the eye one to four times per day according to the routine discretion of a skilled clinician.

In one embodiment of the invention, the compositions are formulated as aqueous pharmaceutical formulations comprising at least one compound of Formula III in an amount of 0.001-2% w/v, and a tonicity agent to maintain a tonicity between 200-400 mOsm/kG, wherein the pH of the formulation is 3-9.

In yet another embodiment, the aqueous pharmaceutical formulation comprises at least one compound of Formula III in an amount of 0.001-2% w/v, one or more complexing and/or solubilizing agents, 0.01-0.5% preservative, 0.01-1% chelating agent, and a tonicity agent to maintain a tonicity between 200-400 mOsm/kG, wherein the pH of the formulation is 4-8. The preferred amount of the compound is 0.01-1% w/v.

The delivery of such ophthalmic preparations may be done using a single unit dose vial wherein the inclusion of a preservative may be precluded. Alternatively, the ophthalmic preparation may be contained in an ophthalmic dropper container intended for multi-use. In such an instance, the multi-use product container may or may not contain a preservative, especially in the event the formulation is self-preserving. Furthermore, the dropper container is designed to deliver a certain fixed volume of product preparation in each drop. The typical drop volume of such an ophthalmic preparation will range from 20-60 μL, preferably 25-55 μL, more preferably 30-50 μL, with 35-50 μL being most preferred.

Glaucoma is an ophthalmic disease that leads to irreversible visual impairment. Primary open-angle glaucoma is characterized by abnormally high resistance to fluid (aqueous humor) drainage from the eye. Cellular contractility and changes in cell-cell and cell-trabeculae adhesion in the trabecular meshwork are major determinants of the resistance to flow. The compounds of the present invention cause a transient, pharmacological perturbation of both cell contractility and cell adhesions, mainly via disruption of the actomyosin-associated cytoskeletal structures and/or the modulation of their interactions with the membrane. Altering the contractility of trabecular meshwork cells leads to drainage-surface expansion. Loss of cell-cell, cell-trabeculae adhesion may influence paracellular fluid flow across Schlemm's canal or alter the fluid flow pathway through the juxtacanalicular tissue of the trabecular meshwork. Both mechanisms likely reduce the resistance of the trabecular meshwork to fluid flow and thereby reduce intraocular pressure in a therapeutically useful manner.

Regulation of the actin cytoskeleton is important in the modulation of fluid transport. Antimitotic drugs markedly interfere with antidiuretic response, strongly implying that cytoskeleton integrity is essential to this function. This role of the cytoskeleton in controlling the epithelial transport is a necessary step in the translocation of the water channel containing particle aggregates and in their delivery to the apical membrane. Osmolality-dependent reorganization of the cytoskeleton and expression of specific stress proteins are important components of the regulatory systems involved in the adaptation of medullary cells to osmotic stress. The compounds of the present invention are useful in directing epithelial function and modulating fluid transport, particularly modulating fluid transport on the ocular surface.

Rho-associated protein kinase inhibitors, due to their regulation of smooth muscle contractility, are useful in the treatment of vasospasm, specifically retinal vasospasm. Relaxation of retinal vasculature increases perfusion rates thereby providing a neuroprotective mechanism (decreased apoptosis and necrosis) in retinal diseases and retinopathies such as glaucoma, ocular hypertension, age-related macular degeneration or retinitis pigmentosa. Additionally, these kinase inhibitors regulate vascular endothelial permeability and as such can play a vasoprotective role to various atherogenic agents.

The present invention provides a method of reducing intraocular pressure, including treating glaucoma such as primary open-angle glaucoma; a method of treating constriction of the visual field; a method of modulating fluid transport on the ocular surface; a method of controlling vasospasm; a method of increasing tissue perfusion; and a method of vasoprotection to atherogenic agents. The method comprises the steps of identifying a subject in need of treatment, and administering to the subject a compound of Formula I or Formula III, in an amount effective to alter the actin cytoskeleton, such as by inhibiting actomyosin interactions.

The present invention is also directed to methods of preventing or treating ocular diseases associated with excessive inflammation, proliferation, remodeling, neurite retraction, corneal neurodegeneration, vaso-permeability and edema. Particularly, this invention relates to methods treating ocular diseases such as allergic conjunctivitis, macular edema, macular degeneration, and blepharitis. The method comprises identifying a subject in need of the treatment, and administering to the subject an effective amount of the compound of Formula III to treat the disease. The subject is a mammalian subject and is preferably a human subject.

In one embodiment, the pharmaceutical composition of the present invention is administered locally to the eye (e.g., topical, intracameral, intravitreal, subretinal, subconjunctival, retrobulbar or via an implant) in the form of ophthalmic formulations.

The compounds of the invention can be combined with ophthalmologically acceptable preservatives, surfactants, viscosity enhancers, penetration enhancers, bioadhesives, antioxidants, buffers, sodium chloride, and water to form an aqueous or non-aqueous, sterile ophthalmic suspension, emulsion, microemulsion, gel, or solution to form the compositions of the invention.

The active compounds disclosed herein can be administered to the eyes of a patient by any suitable means, but are preferably administered by administering a liquid or gel suspension of the active compound in the form of drops, spray or gel. Alternatively, the active compounds can be applied to the eye via liposomes. Further, the active compounds can be infused into the tear film via a pump-catheter system. Another embodiment of the present invention involves the active compound contained within a continuous or selective-release device, for example, membranes such as, but not limited to, those employed in the Ocusert™ System (Alza Corp., Palo Alto, Calif.). As an additional embodiment, the active compounds can be contained within, carried by, or attached to contact lenses that are placed on the eye. Another embodiment of the present invention involves the active compound contained within a swab or sponge that can be applied to the ocular surface. Another embodiment of the present invention involves the active compound contained within a liquid spray that can be applied to the ocular surface. Another embodiment of the present invention involves an injection of the active compound directly into the lacrimal tissues or onto the eye surface.

In addition to the topical administration of the compounds to the eye, the compounds of the invention can be administered systematically by any methods known to a skilled person when used for the purposes described above.

The invention is illustrated further by the following examples that are not to be construed as limiting the invention in scope to the specific procedures described in them.

### EXAMPLES

### Example 1

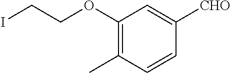

### 3-(2-Iodoethoxy)-4-methylbenzaldehyde

A solution of 3-(2-hydroxyethoxy)-4-methylbenzaldehyde in dichloromethane was cooled to 5° C., and 2.2 equivalents of pyridine and 1.1 equivalents of p-toluenesulfonyl chloride were added. The mixture was allowed to warm to room temperature, and stirred until the reaction is complete as judged by HPLC analysis. The mixture was diluted with additional dichloromethane and washed with dilute aqueous HCl, NaHCO₃, and brine, then evaporated to a residue.

The crude tosylate obtained above was dissolved in acetone, and treated with excess sodium iodide with warming. The reaction was allowed to continue until analysis by HPLC shows the conversion to the iodide is complete, after which the mixture was filtered and evaporated to a residue. Chromotography on silica gel afforded the pure title iodide.

### Example 2

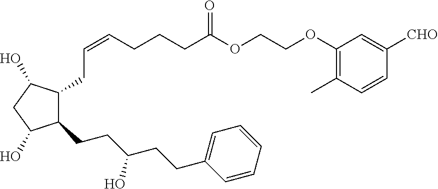 [SMILES: Cc1ccc(C=O)cc1OCCOC(=O)CCC/C=C\C[C@@H]1[C@@H](CC[C@@H](O)CCc2ccccc2)[C@H](O)C[C@@H]1O]

### (Z)-2-(5-Formyl-2-methylphenoxy)ethyl 7-((1R,2R,3R,5S)-3,5-dihydroxy-2-((R)-3-hydroxy-5-phenylpentyl)cyclopentyl)hept-5-enoate

A solution of (Z)-7-((1R,2R,3R,5S)-3,5-dihydroxy-2-((R)-3-hydroxy-5-phenylpentyl)-cyclopentyl)hept-5-enoic acid in DMF was treated with 2 equivalents of 3-(2-iodoethoxy)-4-methylbenzaldehyde and 2 equivalents of DBU, and the mixture warmed to 50° C. The reaction was monitored for conversion to the ester by HPLC. When complete the reaction was cooled, diluted with diethyl ether, and washed with dilute aqueous HCl, NaHCO₃, and brine, and dried over MgSO₄. Evaporation afforded a residue which was chromatographed on silica gel to yield the title ester.

### Example 3

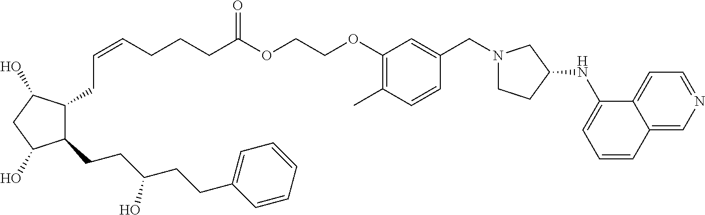 [SMILES: Cc1ccc(CN2CC[C@@H](Nc3cccc4cnccc34)C2)cc1OCCOC(=O)CCC/C=C\C[C@@H]1[C@@H](CC[C@@H](O)CCc2ccccc2)[C@H](O)C[C@@H]1O]

### (Z)-2-(5-(((R)-3-(Isoquinolin-5-ylamino)pyrrolidin-1-yl)methyl)-2-methylphenoxy)ethyl 7-((1R,2R,3R,5S)-3,5-dihydroxy-2-((R)-3-hydroxy-5-phenylpentyl)cyclopentyl)hept-5-enoate

A solution of (R)—N-(pyrrolidin-3-yl)isoquinolin-5-amine and an equimolar amount of (Z)-2-(5-formyl-2-methylphenoxy)ethyl 7-((1R,2R,3R,5S)-3,5-dihydroxy-2-((R)-3-hydroxy-5-phenylpentyl)cyclopentyl)hept-5-enoate in THF was treated with equimolar amounts of glacial acetic acid and sodium triacetoxyborohydride. The reaction was monitored by HPLC for complete conversion of the starting materials to the product, and when complete, was washed with dilute aqueous HCl, NaHCO₃, and brine, and dried over MgSO₄. Evaporation afforded a residue which was chromatographed on silica gel to yield the title compound, represented as (Drug₂-2)-(W-1)-(Link-1)-(Drug₁) within Formula I.

### Example 4

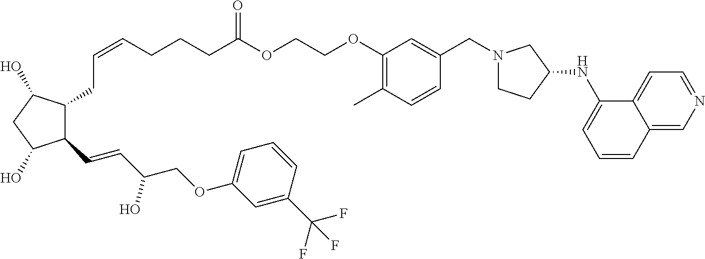 [SMILES: Cc1ccc(CN2CC[C@@H](Nc3cccc4cnccc34)C2)cc1OCCOC(=O)CCC/C=C\C[C@@H]1[C@@H](/C=C/[C@@H](O)COc2cccc(C(F)(F)F)c2)[C@H](O)C[C@@H]1O]

### (Z)-2-(5-(((R)-3-(isoquinolin-5-ylamino)pyrrolidin-1-yl)methyl)-2-methylphenoxy)ethyl 7-((1R,2R,3R,5S)-3,5-dihydroxy-2-((R,E)-3-hydroxy-4-(3-(trifluoromethyl)phenoxy)but-1-enyl)cyclopentyl)hept-5-enoate

A solution of (5Z)-7-((1R,2R,3R,5S)-2-((R,E)-4-(3-(trifluoromethyl)phenoxy)-3-hydroxybut-1-enyl)-3,5-dihydroxycyclopentyl)hept-5-enoic acid in DMF is treated with 2 equivalents of 3-(2-iodoethoxy)-4-methylbenzaldehyde and 2 equivalents of DBU, and the mixture is warmed to 50° C. The reaction is monitored for conversion to the ester by HPLC. When complete the reaction is cooled, diluted with diethyl ether, and washed with dilute aqueous HCl, NaHCO₃, and brine, and is dried over MgSO₄. Evaporation affords a residue which is chromatographed on silica gel to yield the intermediate formyl ester, (5Z)-2-(5-formyl-2-methylphenoxy)ethyl 7-((1R,2R,3R,5S)-2-((R,E)-4-(3-(trifluoromethyl)phenoxy)-3-hydroxybut-1-enyl)-3,5-dihydroxycyclopentyl)hept-5-enoate. A solution of (R)—N-(pyrrolidin-3-yl)isoquinolin-5-amine and an equimolar amount of the intermediate formyl ester in THF is treated with equimolar amounts of glacial acetic acid and sodium triacetoxyborohydride. The reaction is monitored by HPLC for complete conversion of the starting materials to the product, and when complete, is washed with dilute aqueous HCl, NaHCO₃, and brine, and is dried over MgSO₄. Evaporation affords a residue which is chromatographed on silica gel to yield the title compound, represented as (Drug₂-2)-(W-2)-(Link-1)-(Drug₁) within Formula I.

### Example 5

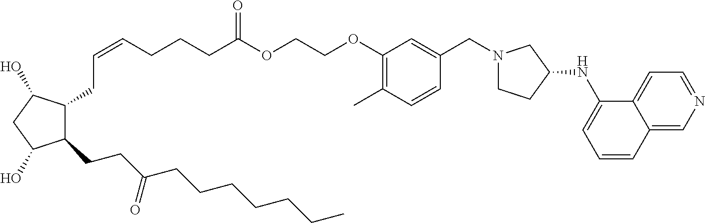 [SMILES: CCCCCCCC(=O)CC[C@@H]1[C@@H](C/C=C\CCCC(=O)OCCOc2cc(CN3CC[C@@H](Nc4cccc5cnccc45)C3)ccc2C)[C@@H](O)C[C@H]1O]

### (Z)-2-(5-(((R)-3-(isoquinolin-5-ylamino)pyrrolidin-1-yl)methyl)-2-methylphenoxy)ethyl 7-((1R,2R,3R,5S)-3,5-dihydroxy-2-(3-oxodecyl)cyclopentyl)hept-5-enoate

A solution of (5Z)-7-((1R,2R,3R,5S)-3,5-dihydroxy-2-(3-oxodecyl)cyclopentyl)hept-5-enoic acid in DMF is treated with 2 equivalents of 3-(2-iodoethoxy)-4-methylbenzaldehyde and 2 equivalents of DBU, and the mixture is warmed to 50° C. The reaction is monitored for conversion to the ester by HPLC. When complete the reaction is cooled, diluted with diethyl ether, and washed with dilute aqueous HCl, NaHCO₃, and brine, and is dried over MgSO₄. Evaporation affords a residue which is chromatographed on silica gel to yield the intermediate formyl ester, (5Z)-2-(5-formyl-2-methylphenoxy)ethyl 7-((1R,2R,3R,5S)-3,5-dihydroxy-2-(3-oxodecyl)cyclopentyl)hept-5-enoate. A solution of (R)—N-(pyrrolidin-3-yl)isoquinolin-5-amine and an equimolar amount of the intermediate formyl ester in THF is treated with equimolar amounts of glacial acetic acid and sodium triacetoxyborohydride. The reaction is monitored by HPLC for complete conversion of the starting materials to the product, and when complete, is washed with dilute aqueous HCl, NaHCO₃, and brine, and is dried over MgSO₄. Evaporation affords a residue which is chromatographed on silica gel to yield the title compound, represented as (Drug₂-2)-(W-4)-(Link-1)-(Drug₁) within Formula I.

### Example 6

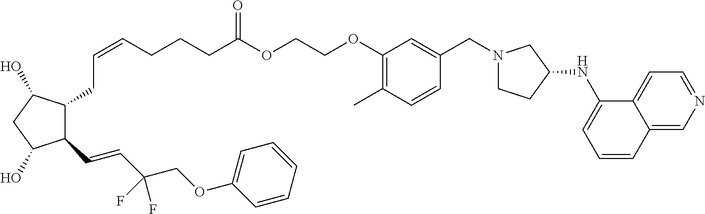 [SMILES: Cc1ccc(CN2CC[C@@H](Nc3cccc4cnccc34)C2)cc1OCCOC(=O)CCC/C=C\C[C@@H]1[C@@H](/C=C/C(F)(F)COc2ccccc2)[C@H](O)C[C@@H]1O]

### (Z)-2-(5-(((R)-3-(isoquinolin-5-ylamino)pyrrolidin-1-yl)methyl)-2-methylphenoxy)ethyl 7-((1R,2R,3R,5S)-2-(E)-3,3-difluoro-4-phenoxybut-1-enyl)-3,5-dihydroxycyclopentyl)hept-5-enoate

A solution of (5Z)-7-((1R,2R,3R,5S)-2-((E)-3,3-difluoro-4-phenoxybut-1-enyl)-3,5-dihydroxycyclopentyl)hept-5-enoic acid in DMF is treated with 2 equivalents of 3-(2-iodoethoxy)-4-methylbenzaldehyde and 2 equivalents of DBU, and the mixture is warmed to 50° C. The reaction is monitored for conversion to the ester by HPLC. When complete the reaction is cooled, diluted with diethyl ether, and washed with dilute aqueous HCl, NaHCO₃, and brine, and is dried over MgSO₄. Evaporation affords a residue which is chromatographed on silica gel to yield the intermediate formyl ester, (5Z)-2-(5-formyl-2-methylphenoxy)ethyl 7-((1R,2R,3R,5S)-2-((E)-3,3-difluoro-4-phenoxybut-1-enyl)-3,5-dihydroxycyclopentyl)hept-5-enoate. A solution of (R)—N-(pyrrolidin-3-yl)isoquinolin-5-amine and an equimolar amount of the intermediate formyl ester in THF is treated with equimolar amounts of glacial acetic acid and sodium triacetoxyborohydride. The reaction is monitored by HPLC for complete conversion of the starting materials to the product, and when complete, is washed with dilute aqueous HCl, NaHCO₃, and brine, and is dried over MgSO₄. Evaporation affords a residue which is chromatographed on silica gel to yield the title compound, represented as (Drug₂-2)-(W-3)-(Link-1)-(Drug₁) within Formula I.

### Example 7

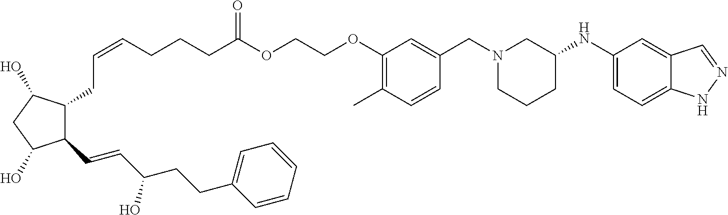 [SMILES: Cc1ccc(CN2CCC[C@@H](Nc3ccc4[nH]ncc4c3)C2)cc1OCCOC(=O)CCC/C=C\C[C@@H]1[C@@H](/C=C/[C@@H](O)CCc2ccccc2)[C@H](O)C[C@@H]1O]

### (5Z)-2-(5-(OR)-3-(1H-indazol-5-ylamino)piperidin-1-yl)methyl)-2-methylphenoxy)ethyl 7-((1R,2R,3R,5S)-3,5-dihydroxy-2-((S,E)-3-hydroxy-5-phenylpent-1-enyl)cyclopentyl)hept-5-enoate

A solution of (5Z)-7-((1R,2R,3R,5S)-3,5-dihydroxy-2-((S,E)-3-hydroxy-5-phenylpent-1-enyl)cyclopentyl)hept-5-enoic acid in DMF is treated with 2 equivalents of 3-(2-iodoethoxy)-4-methylbenzaldehyde and 2 equivalents of DBU, and the mixture is warmed to 50° C. The reaction is monitored for conversion to the ester by HPLC. When complete the reaction is cooled, diluted with diethyl ether, and washed with dilute aqueous HCl, NaHCO₃, and brine, and is dried over MgSO₄. Evaporation affords a residue which is chromatographed on silica gel to yield the intermediate formyl ester, (5Z)-2-(5-formyl-2-methylphenoxy)ethyl 7-((1R,2R,3R,5S)-3,5-dihydroxy-2-((S,E)-3-hydroxy-5-phenylpent-1-enyl)cyclopentyl)hept-5-enoate. A solution of N—((R)-piperidin-3-yl)-1H-indazol-5-amine and an equimolar amount of the intermediate formyl ester in THF is treated with equimolar amounts of glacial acetic acid and sodium triacetoxyborohydride. The reaction is monitored by HPLC for complete conversion of the starting materials to the product, and when complete, is washed with dilute aqueous HCl, NaHCO₃, and brine, and is dried over MgSO₄. Evaporation affords a residue which is chromatographed on silica gel to yield the title compound, represented as (Drug₂-2)-(W-5)-(Link-1)-(Drug₁) within Formula I.

### Example 8

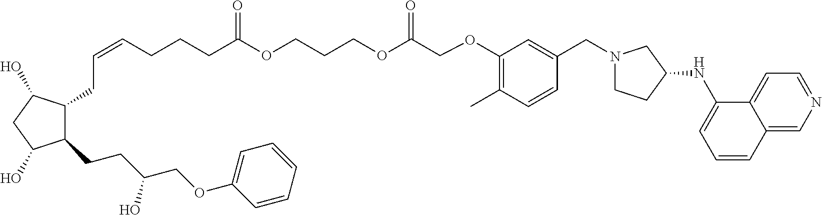 [SMILES: Cc1ccc(CN2CC[C@@H](Nc3cccc4cnccc34)C2)cc1OCC(=O)OCCCOC(=O)CCC/C=C\C[C@@H]1[C@@H](CC[C@@H](O)COc2ccccc2)[C@H](O)C[C@@H]1O]

### (Z)-3-(2-(5-(((R)-3-(isoquinolin-5-ylamino)pyrrolidin-1-yl)methyl)-2-methylphenoxy)acetoxy)propyl 7-((1R,2R,3R,5S)-3,5-dihydroxy-2-((R)-3-hydroxy-5-phenylpentyl)cyclopentyl)hept-5-enoate

A solution of 2-(5-formyl-2-methylphenoxy)acetic acid in DMF is treated with 1.5 equivalents of dicyclohexylcarbodiimide, 2 equivalents of 3-bromopropanol, and a catalytic amount of 4-N,N-dimethylaminopyridine at 0° C. then is warmed to 50° C. The reaction is monitored for conversion to the bromoester by HPLC. When complete the reaction is cooled, diluted with diethyl ether, and washed with dilute aqueous HCl, NaHCO₃, and brine, and is dried over MgSO₄. Evaporation affords a residue which is chromatographed on silica gel to yield the intermediate bromide, 3-bromopropyl 2-(5-formyl-2-methylphenoxy)acetate. A solution of (Z)-7-((1R,2R,3R,5S)-3,5-dihydroxy-2-((R)-3-hydroxy-5-phenylpentyl)-cyclopentyl)hept-5-enoic acid in DMF is treated with 2 equivalents of the intermediate bromide and 2 equivalents of DBU, and the mixture is warmed to 50° C. The reaction is monitored for conversion to the ester by HPLC. When complete the reaction is cooled, diluted with diethyl ether, and washed with dilute aqueous HCl, NaHCO₃, and brine, and is dried over MgSO₄. Evaporation affords a residue which is chromatographed on silica gel to yield the intermediate formyl ester, (Z)-3-(2-(5-formyl-2-methylphenoxy)acetoyloxy)propyl 7-((1R,2R,3R,5S)-3,5-dihydroxy-2-((R)-3-hydroxy-5-phenylpentyl)cyclopentyl)hept-5-enoate. A solution of (R)—N-(pyrrolidin-3-yl)isoquinolin-5-amine and an equimolar amount of the intermediate formyl ester in THF is treated with equimolar amounts of glacial acetic acid and sodium triacetoxyborohydride. The reaction is monitored by HPLC for complete conversion of the starting materials to the product, and when complete, is washed with dilute aqueous HCl, NaHCO₃, and brine, and is dried over MgSO₄. Evaporation affords a residue which is chromatographed on silica gel to yield the title compound, represented as (Drug₂-2)-(W-1)-(Link-2)-(Drug₁) within Formula I.

### Example 9

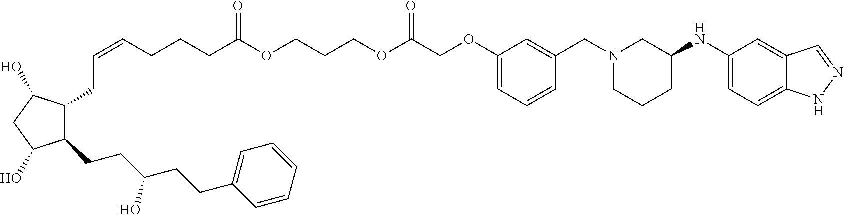 [SMILES: O=C(CCC/C=C\C[C@@H]1[C@@H](CC[C@@H](O)CCc2ccccc2)[C@H](O)C[C@@H]1O)OCCCOC(=O)COc1cccc(CN2CCC[C@H](Nc3ccc4[nH]ncc4c3)C2)c1]

### (Z)-3-(2-(3-(((S)-3-(1H-indazol-5-ylamino)piperidin-1-yl)methyl)phenoxy)acetoxy)propyl 7-((1R,2R,3R,5S)-3,5-dihydroxy-2-((R)-3-hydroxy-5-phenylpentyl)cyclopentyl)hept-5-enoate

A solution of 2-(3-formylphenoxy)acetic acid in DMF is treated with 1.5 equivalents of dicyclohexylcarbodiimide, 2 equivalents of 3-bromopropanol, and a catalytic amount of 4-N,N-dimethylaminopyridine at 0° C. then is warmed to 50° C. The reaction is monitored for conversion to the bromoester by HPLC. When complete the reaction is cooled, diluted with diethyl ether, and washed with dilute aqueous HCl, NaHCO₃, and brine, and is dried over MgSO₄. Evaporation affords a residue which is chromatographed on silica gel to yield the intermediate bromide, 3-bromopropyl 2-(3-formylphenoxy)acetate. A solution of (Z)-7-((1R,2R,3R,5S)-3,5-dihydroxy-2-((R)-3-hydroxy-5-phenylpentyl)-cyclopentyl)hept-5-enoic acid in DMF is treated with 2 equivalents of the intermediate bromide and 2 equivalents of DBU, and the mixture is warmed to 50° C. The reaction is monitored for conversion to the ester by HPLC. When complete the reaction is cooled, diluted with diethyl ether, and washed with dilute aqueous HCl, NaHCO₃, and brine, and is dried over MgSO₄. Evaporation affords a residue which is chromatographed on silica gel to yield the intermediate formyl ester, (Z)-3-(2-(3-formylphenoxy)acetoyloxy)propyl 7-((1R,2R,3R,5S)-3,5-dihydroxy-2-((R)-3-hydroxy-5-phenylpentyl)cyclopentyl)hept-5-enoate. A solution of N—((R)-piperidin-3-yl)-1H-indazol-5-amine and an equimolar amount of the intermediate formyl ester in THF is treated with equimolar amounts of glacial acetic acid and sodium triacetoxyborohydride. The reaction is monitored by HPLC for complete conversion of the starting materials to the product, and when complete, is washed with dilute aqueous HCl, NaHCO₃, and brine, and is dried over MgSO₄. Evaporation affords a residue which is chromatographed on silica gel to yield the title compound, represented as (Drug₂-2)-(W-1)-(Link-2)-(Drug₁) within Formula I.

### Example 10

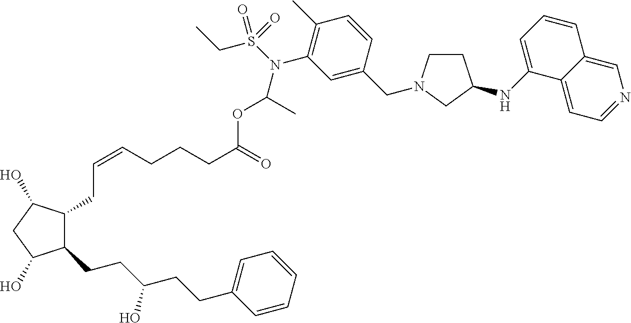 [SMILES: CCS(=O)(=O)N(c1cc(CN2CC[C@@H](Nc3cccc4cnccc34)C2)ccc1C)C(C)OC(=O)CCC/C=C\C[C@@H]1[C@@H](CC[C@@H](O)CCc2ccccc2)[C@H](O)C[C@@H]1O]

### (Z)-1-(N-(5-(((R)-3-(isoquinolin-5-ylamino)pyrrolidin-1-yl)methyl)-2-methylphenyl)ethylsulfonamido)ethyl 7-((1R,2R,3R,5S)-3,5-dihydroxy-2-((R)-3-hydroxy-5-phenylpentyl)cyclopentyl)hept-5-enoate

A solution of (Z)-7-((1R,2R,3R,5S)-3,5-dihydroxy-2-((R)-3-hydroxy-5-phenylpentyl)-cyclopentyl)hept-5-enoic acid in DMF is treated with 2 equivalents of 1-iodo-1-bromoethane and 2 equivalents of DBU, and the mixture is warmed to 50° C. The reaction is monitored for conversion to the ester by HPLC. When complete the reaction is cooled, diluted with diethyl ether, and washed with dilute aqueous HCl, NaHCO₃, and brine, and dried over MgSO₄. Evaporation affords a residue which is chromatographed on silica gel to yield the intermediate ester, (Z)-1-bromoethyl 7-((1R,2R,3R,5S)-3,5-dihydroxy-2-((R)-3-hydroxy-5-phenylpentyl)cyclopentyl)hept-5-enoate. A solution of (R)—N-(3-((3-(isoquinolin-5-ylamino)pyrrolidin-1-yl)methyl)-6-methylphenyl)methanesulfonamide (prepared according to WO 2008/077057) in toluene is treated with 2 equivalents of the intermediate ester and 2 equivalents of potassium carbonate. The mixture is refluxed and monitored by HPLC for complete conversion of the starting materials to the product, and when complete, is washed with dilute aqueous HCl, NaHCO₃, and brine, and is dried over MgSO₄. Evaporation affords a residue which is chromatographed on silica gel to yield the title compound, represented as (Drug₂-2)-(W-1)-(Link-3)-(Drug₁) within Formula I.

### Example 11

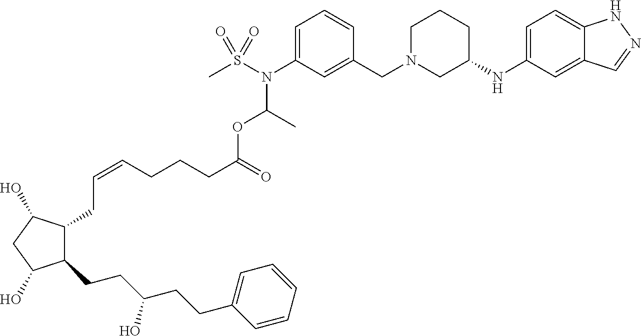 [SMILES: CC(OC(=O)CCC/C=C\C[C@@H]1[C@@H](CC[C@@H](O)CCc2ccccc2)[C@H](O)C[C@@H]1O)N(c1cccc(CN2CCC[C@H](Nc3ccc4[nH]ncc4c3)C2)c1)S(C)(=O)=O]

### (Z)-1-(N-(3-(((S)-3-(1H-indazol-5-ylamino)piperidin-1-yl)methyl)phenyl)methylsulfonamido)ethyl 7-((1R,2R,3R,5S)-3,5-dihydroxy-2-((R)-3-hydroxy-5-phenylpentyl)cyclopentyl)hept-5-enoate

A solution of (Z)-7-((1R,2R,3R,5S)-3,5-dihydroxy-2-((R)-3-hydroxy-5-phenylpentyl)-cyclopentyl)hept-5-enoic acid in DMF is treated with 2 equivalents of 1-iodo-1-bromoethane and 2 equivalents of DBU, and the mixture is warmed to 50° C. The reaction is monitored for conversion to the ester by HPLC. When complete the reaction is cooled, diluted with diethyl ether, and washed with dilute aqueous HCl, NaHCO₃, and brine, and dried over MgSO₄. Evaporation affords a residue which is chromatographed on silica gel to yield the intermediate ester, (Z)-1-bromoethyl 7-((1R,2R,3R,5S)-3,5-dihydroxy-2-((R)-3-hydroxy-5-phenylpentyl)cyclopentyl)hept-5-enoate. A solution of N-(3-(((S)-3-(1H-indazol-5-ylamino)piperidin-1-yl)methyl)phenyl)methylsulfonamide (prepared according to WO 2008/077057) in toluene is treated with 2 equivalents of the intermediate ester and 2 equivalents of potassium carbonate. The mixture is refluxed and monitored by HPLC for complete conversion of the starting materials to the product, and when complete, is washed with dilute aqueous HCl, NaHCO₃, and brine, and is dried over MgSO₄. Evaporation affords a residue which is chromatographed on silica gel to yield the title compound, represented as (Drug₂-2)-(W-1)-(Link-3)-(Drug₁) within Formula I.

### Example 12

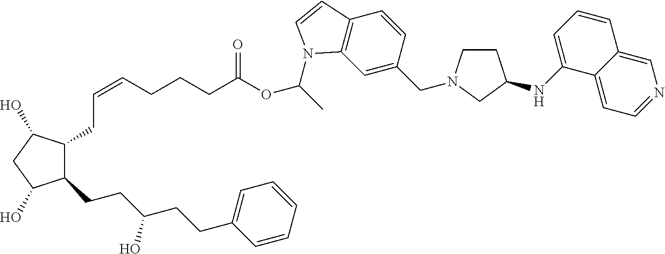 [SMILES: CC(OC(=O)CCC/C=C\C[C@@H]1[C@@H](CC[C@@H](O)CCc2ccccc2)[C@H](O)C[C@@H]1O)n1ccc2ccc(CN3CC[C@@H](Nc4cccc5cnccc45)C3)cc21]

### (Z)-1-(6-(((R)-3-(isoquinolin-5-ylamino)pyrrolidin-1-yl)methyl)-1H-indol-1-yl)ethyl 7-((1R,2R,3R,5S)-3,5-dihydroxy-2-((R)-3-hydroxy-5-phenylpentyl)cyclopentyl)hept-5-enoate

A solution of (Z)-7-((1R,2R,3R,5S)-3,5-dihydroxy-2-((R)-3-hydroxy-5-phenylpentyl)-cyclopentyl)hept-5-enoic acid in DMF is treated with 2 equivalents of 1-iodo-1-bromoethane and 2 equivalents of DBU, and the mixture is warmed to 50° C. The reaction is monitored for conversion to the ester by HPLC. When complete the reaction is cooled, diluted with diethyl ether, and washed with dilute aqueous HCl, NaHCO₃, and brine, and dried over MgSO₄. Evaporation affords a residue which is chromatographed on silica gel to yield the intermediate ester, (Z)-1-bromoethyl 7-((1R,2R,3R,5S)-3,5-dihydroxy-2-((R)-3-hydroxy-5-phenylpentyl)cyclopentyl)hept-5-enoate. A solution of 6-(((R)-3-(isoquinolin-5-ylamino)pyrrolidin-1-yl)methyl)-1H-indole (prepared according to WO 2008/077057) in toluene is treated with 2 equivalents of the intermediate ester and 2 equivalents of potassium carbonate. The mixture is refluxed and monitored by HPLC for complete conversion of the starting materials to the product, and when complete, is washed with dilute aqueous HCl, NaHCO₃, and brine, and is dried over MgSO₄. Evaporation affords a residue which is chromatographed on silica gel to yield the title compound, represented as (Drug₂-2)-(W-1)-(Link-3)-(Drug₁) within Formula I.

### Example 13

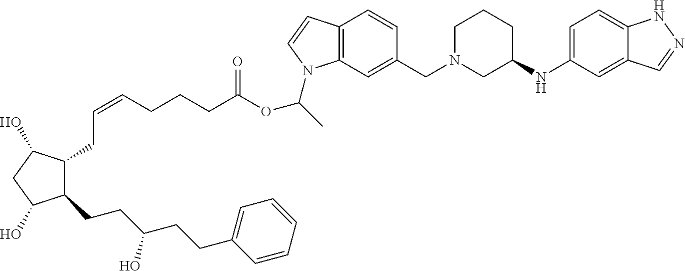 [SMILES: CC(OC(=O)CCC/C=C\C[C@@H]1[C@@H](CC[C@@H](O)CCc2ccccc2)[C@H](O)C[C@@H]1O)n1ccc2ccc(CN3CCC[C@@H](Nc4ccc5[nH]ncc5c4)C3)cc21]

(Z)-1-(6-(((R)-3-(1H-indazol-5-ylamino)piperidin-1-yl)methyl)-1H-indol-1-yl)ethyl 7-((1R,2R,3R,5S)-3,5-dihydroxy-2-((R)-3-hydroxy-5-phenylpentyl)cyclopentyl)hept-5-enoate

A solution of (Z)-7-((1R,2R,3R,5S)-3,5-dihydroxy-2-((R)-3-hydroxy-5-phenylpentyl)-cyclopentyl)hept-5-enoic acid in DMF is treated with 2 equivalents of 1-iodo-1-bromoethane and 2 equivalents of DBU, and the mixture is warmed to 50° C. The reaction is monitored for conversion to the ester by HPLC. When complete the reaction is cooled, diluted with diethyl ether, and washed with dilute aqueous HCl, NaHCO₃, and brine, and dried over MgSO₄. Evaporation affords a residue which is chromatographed on silica gel to yield the intermediate ester, (Z)-1-bromoethyl 7-((1R,2R,3R,5S)-3,5-dihydroxy-2-((R)-3-hydroxy-5-phenylpentyl)cyclopentyl)hept-5-enoate. A solution of 6-(((R)-3-(1H-indazol-5-ylamino)piperidin-1-yl)methyl)-1H-indole (as prepared in WO 2008/077057) in toluene is treated with 2 equivalents of the intermediate ester and 2 equivalents of potassium carbonate. The mixture is refluxed and monitored by HPLC for complete conversion of the starting materials to the product, and when complete, is washed with dilute aqueous HCl, NaHCO₃, and brine, and is dried over MgSO₄. Evaporation affords a residue which is chromatographed on silica gel to yield the title compound, represented as (Drug₂-2)-(W-1)-(Link-3)-(Drug₁) within Formula I.

### Example 14

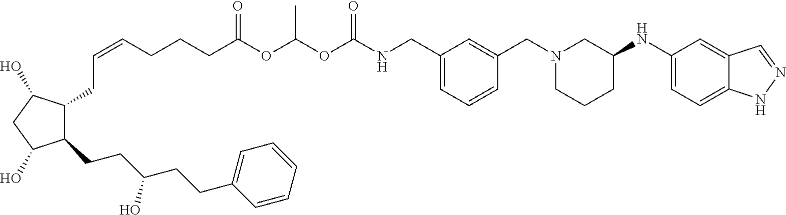 [SMILES: CC(OC(=O)CCC/C=C\C[C@@H]1[C@@H](CC[C@@H](O)CCc2ccccc2)[C@H](O)C[C@@H]1O)OC(=O)NCc1cccc(CN2CCC[C@H](Nc3ccc4[nH]ncc4c3)C2)c1]

### (Z)-1-(3-(((S)-3-(1H-indazol-5-ylamino)piperidin-1-yl)methyl)benzylcarbamoyloxy)ethyl 7-((1R,2R,3R,5S)-3,5-dihydroxy-2-((R)-3-hydroxy-5-phenylpentyl)cyclopentyl)hept-5-enoate

A solution of 3-(aminomethyl)benzaldehyde in pyridine is treated with 2 equivalents of 1-chloroethyl chloroformate. The reaction is monitored for conversion to the carbamate by HPLC. When complete the reaction is evaporated and the residue is dissolved in chloroform and washed with dilute HCl, NaHCO₃, and brine and is dried over MgSO₄. Evaporation affords a residue which is chromatographed on silica gel to yield the intermediate carbamate, 1-chloroethyl 3-formylbenzylcarbamate. A solution of (Z)-7-((1R,2R,3R,5S)-3,5-dihydroxy-2-((R)-3-hydroxy-5-phenylpentyl)-cyclopentyl)hept-5-enoic acid in DMF is treated with 2 equivalents of the intermediate carbamate and 2 equivalents of DBU, and the mixture is warmed to 50° C. The reaction is monitored for conversion to the ester acetal carbamate by HPLC. When complete the reaction is cooled, diluted with diethyl ether, and washed with dilute aqueous HCl, NaHCO₃, and brine, and dried over MgSO₄. Evaporation affords a residue which is chromatographed on silica gel to yield the intermediate ester acetal carbamate, 1-((Z)-7-((1R,2R,3R,5S)-3,5-dihydroxy-2-((R)-3-hydroxy-5-phenylpentyl)cyclopentyl)hept-5-enoyloxy)ethyl 3-formylbenzylcarbamate. A solution of N—((R)-piperidin-3-yl)-1H-indazol-5-amine and an equimolar amount of the intermediate ester acetal carbamate in THF is treated with equimolar amounts of glacial acetic acid and sodium triacetoxyborohydride. The reaction is monitored by HPLC for complete conversion of the starting materials to the product, and when complete, is washed with dilute aqueous HCl, NaHCO₃, and brine, and is dried over MgSO₄. Evaporation affords a residue which is chromatographed on silica gel to yield the title compound, represented as (Drug₂-2)-(W-1)-(Link-4)-(Drug₁) within Formula I.

### Example 15

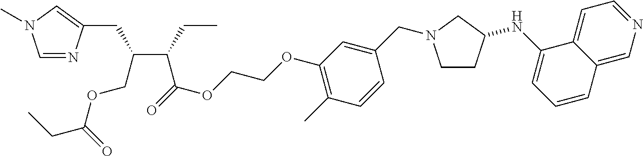 [SMILES: CCC(=O)OC[C@H](Cc1cn(C)cn1)[C@H](CC)C(=O)OCCOc1cc(CN2CC[C@@H](Nc3cccc4cnccc34)C2)ccc1C]

### (2S,3R)-2-(5-(((R)-3-(isoquinolin-5-ylamino)pyrrolidin-1-yl)methyl)-2-methylphenoxy)ethyl 2-ethyl-4-(1-methyl-1H-imidazol-4-yl)-3-(propionyloxymethyl)butanoate

A solution of (2S,3R)-2-ethyl-3-((1-methyl-1H-imidazol-4-yl)methyl)-4-(propionyloxy)butanoic acid in DMF is treated with 2 equivalents of 3-(2-iodoethoxy)-4-methylbenzaldehyde and 2 equivalents of DBU, and the mixture is warmed to 50° C. The reaction is monitored for conversion to the ester by HPLC. When complete the reaction is cooled, diluted with diethyl ether, and washed with dilute aqueous HCl, NaHCO₃, and brine, and is dried over MgSO₄. Evaporation affords a residue which is chromatographed on silica gel to yield the intermediate formyl ester, (2S,3R)-2-(5-formyl-2-methylphenoxy)ethyl 2-ethyl-3-((1-methyl-1H-imidazol-4-yl)methyl)-4-(propionyloxy)butanoate). A solution of (R)—N-(pyrrolidin-3-yl)isoquinolin-5-amine and an equimolar amount of the intermediate formyl ester in THF is treated with equimolar amounts of glacial acetic acid and sodium triacetoxyborohydride. The reaction is monitored by HPLC for complete conversion of the starting materials to the product, and when complete, is washed with dilute aqueous HCl, NaHCO₃, and brine, and is dried over MgSO₄. Evaporation affords a residue which is chromatographed on silica gel to yield the title compound, represented as (Drug₂-1)-(Link-1)-(Drug₁) within Formula I.

### Rho Kinase Inhibition Assay

Inhibition of ROCK2 activity is determined using the IMAP™ Screening Express Kit (Molecular Devices product number #8073). ROCK2 kinase (UpstateChemicon #14-451) and Fluorescein tagged substrate peptide Fl-AKRRRLSSLRA (Molecular Devices product number R7184) is preincubated with test compound for 5 minutes in buffer containing 10 mM Tris-HCl pH 7.2, 10 mM MgCl₂, and 0.1% BSA. Following the preincubation, 10 μM ATP is added to initiate the reaction. After 60 minutes at room temperature, Molecular Devices IMAP™ binding solution is added to bind phosphorylated substrate. After 30 minutes of incubation in the presence of the IMAP™ beads the fluorescence polarization is read and the ratio is reported as mP. IC₅₀ results are calculated using the Prism software from Graphpad.

This assay demonstrates a compound's ability to inhibit ROCK2 in an in vitro setting using the isolated enzyme. Compounds having ROCK2 IC₅₀ values on the order of 2 μM or below have been shown to possess efficacy in numerous studies using in vivo models of the disease processes described in this application, specifically in models of elevated IOP and glaucoma. See Tian et al., Arch. Ophthalmol. 116: 633-643, 1998; Tian et al., Invest. Ophthalmol. Vis. Sci. 40: 239-242, 1999; Tian, et al., Exp. Eye Res. 68: 649-655; 1999; Sabanay, et al., Arch. Ophthalmol. 118: 955-962, 2000; Volberg, et al., Cell Motil. Cytoskel. 29: 321-338, 1994; Tian, et al., Exp. Eye Res. 71: 551-566, 2000; Tokushige, et al., Invest. Ophthalmol. Vis. Sci. 48: 3216-3222, 2007; Honjo, et al., Invest. Ophthalmol. Vis. Sci. 42: 137-144, 2001.

### NIH/3T3 Cell Morphology Assay

NIH/3T3 cells are grown in DMEM-H containing glutamine and 10% Colorado Calf Serum. Cells are passaged regularly prior to reaching confluence. Eighteen to 24 hours prior to experimentation, the cells are plated onto Poly-L-Lysine-coated glass bottom 24-well plates. On the day of experimentation, the cell culture medium is removed and is replaced with the same medium containing from 10 nM to 25 μM of the test compound, and the cells are incubated for 60 minutes at 37° C. The culture medium is then removed and the cells are washed with warmed PBS and fixed for 10 minutes with warmed 4% paraformaldehyde. The cells are permeabilized with 0.5% Triton-X, stained with TRITC-conjugated phalloidin and imaged using a Nikon Eclipse E600 epifluorescent microscope to determine the degree of actin disruption. Results are expressed as a numerical score indicating the observed degree of disruption of the actin cytoskeleton at the test concentration, ranging from 0 (no effect) to 4 (complete disruption), and are the average of at least 2 determinations.

The assay demonstrates that a compound's in vitro ROCK inhibition activity can manifest itself in morphology changes, such as actin stress fiber disassembly and alteration in focal adhesions in intact cells leading to inhibition of acto-myosin driven cellular contraction. These morphology changes are thought to provide the basis for the beneficial pharmacological effects sought in the setting of the disease processes described in this application, specifically the lowering of elevated IOP in hypertensive eyes via increased outflow through the trabecular meshwork.

### Ocular Pharmacokinetic Assay

Intraocular fluid (aqueous humor) is collected from New Zealand White rabbits to determine corneal and anterior chamber pharmacokinetics of formulations containing test compounds of interest. Each animal is dosed bilaterally with 2×10 μl of 25 mM of each test compound (in 10 mM acetate buffered saline, 0.01% benzalkonium chloride, 0.05% EDTA, pH 4.5) or with vehicle. During instillation, the upper and lower eyelids are immobilized and the compound is administered to the superior aspect of the globe allowing it to flow across the ocular surface. Following instillation, blinking is prevented for 30 seconds. Aqueous humor is collected from 30 minutes to 8 hours following topical instillation using a 30-gauge needle inserted proximal to the corneal scleral limbus. Subsequently 30 μl of aqueous humor is aspirated using a 300 μl syringe. Aqueous humor samples are assayed for the concentration of the test compound using an LC/MS/MS assay system. All experiments are conducted in accordance with the ARVO Statement for the Use of Animals in Ophthalmic and Vision Research and in compliance with National Institutes of Health.

This pharmacokinetic assay shows that the compounds of the invention when dosed topically are able to penetrate the eye and achieve concentrations in the aqueous humor adequate to provide substantial ROCK inhibition at the sight of action, that is, concentrations at or above the ROCK IC₅₀ of the compound in question. Further, it shows that these compounds can show different pharmacokinetic profiles on topical ocular dosing.

The invention, and the manner and process of making and using it, are now described in such full, clear, concise and exact terms as to enable any person skilled in the art to which it pertains, to make and use the same. It is to be understood that the foregoing describes preferred embodiments of the present invention and that modifications may be made therein without departing from the scope of the present invention as set forth in the claims. To particularly point out and distinctly claim the subject matter regarded as invention, the following claims conclude this specification.

What is claimed is:

1. A compound of Formula III, or its pharmaceutically acceptable salt or solvate, 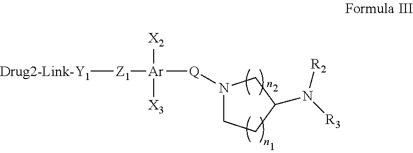
Q is C═O, SO₂, or (CR₄R₅)ₙ₃;
n₁ is 1, 2, or 3;
n₂ is 1 or 2;
n₃ is 0, 1, 2, or 3;
wherein the ring represented by 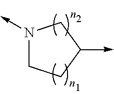
is optionally substituted by alkyl, halo, oxo, OR₆, NR₆R₇, or SR₆;
R₂ is selected from the following heteroaryl systems, optionally substituted: 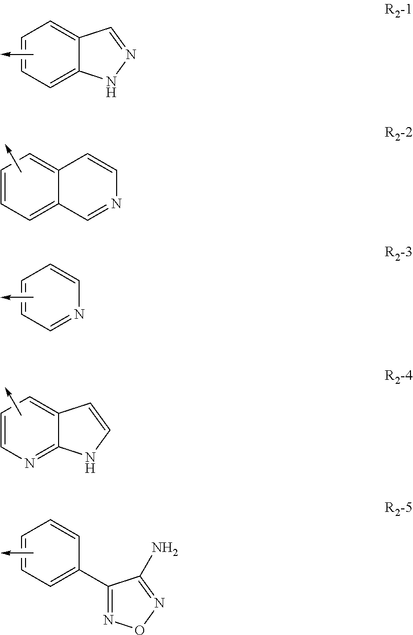
R₃-R₇ are independently H, alkyl, alkenyl, alkynyl, cycloalkyl, cycloalkenyl, cycloalkylalkyl, cycloalkylalkenyl, or cycloalkylalkynyl optionally substituted;
Ar is a monocyclic aryl, bicyclic aryl, monocyclic heteroaryl, or bicyclic heteroaryl;
X₂ and X₃ are either absent, or are substituents on Ar and independently in the form Y₂—Z₂ and Y₃—Z₃ in which Z₂ and Z₃ are attached to Ar;
Y₁ is O, CO₂, NR_{B}, SO₂NR₈, NR₈SO₂, NR₈CO, or N-containing heteroaryl;
Y₂ and Y₃ are independently selected from the group consisting of: H, halogen, OR₈, NR₈R₉, NO₂, SR₈, SOR₈, SO₂R₈, SO₂NR₈R₉, NR₈SO₂R₉, OCF₃, CO₂R₈, CONR₈R₉, NR₈C(═O)R₉, NR₈C(═O)OR₉, OC(═O)NR₈R₉, NR₈C(═O)NR₉R₁₀, N-containing heterocycle, and N-containing heteroaryl;
Z₁, Z₂, and Z₃ are independently selected from the group consisting of: alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, heterocycle, (heterocycle)alkyl, (heterocycle)alkenyl, (heterocycle)alkynyl, and absent;
R₈-R₁₀ are independently selected from the group consisting of: absent, H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heterocycle, heteroaryl, heteroarylalkyl, heteroarylalkenyl, heteroarylalkynyl, (heterocycle)alkyl, (heterocycle)alkenyl, (heterocycle)alkynyl, or heterocycle; optionally substituted by OR₁₁, COOR₁₁, NR₁₁R₁₂, NO₂, SR₁₁, SOR₁₁, SO₂R₁₁, SO₂NR₁₁R₁₂, NR₁₁SO₂R₁₂, OCF₃, CONR₁₁R₁₂, NR₁₁C(═O)R₁₂, NR₁₁C(═O)OR₁₂, OC(═O)NR₁₁R₁₂, and NR₁₁C(═O)NR₁₂R₁₃;
with any two of the groups R₈, R₉ and R₁₀ being optionally joined with a link selected from the group consisting of bond, —O—, —S—, —SO—, —SO₂—, and —NR₁₁— to form a ring;
R₁₁-R₁₃ are independently selected from the group consisting of: H, alkyl, alkenyl, alkynyl, aryl, arylalkyl, arylalkenyl, arylalkynyl, cycloalkyl, cycloalkenyl, cycloalkylalkyl, cycloalkylalkenyl, cycloalkylalkynyl, heteroaryl, heteroarylalkyl, heteroarylalkenyl, heteroarylalkynyl, (heterocycle)alkyl, (heterocycle)alkenyl, (heterocycle)alkynyl, heterocycle, and absent;
Link is selected from groups consisting of:
Link-1: Absent
Link-2: 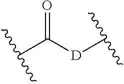
Link-3: 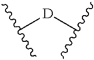
Link-4: 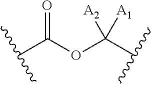
wherein A₁ and A₂ are independently hydrogen, alkyl, or arylalkyl, optionally substituted;
and A₁ and A₂ are optionally joined to form a ring through a direct bond or through a bond to a nitrogen, oxygen, or sulfur atom;
D is alkyl, alkenyl, aryl, arylalkyl, arylalkenyl, heteroaryl, heteroarylalkyl, heteroarylalkenyl, cycloalkyl, cycloalkenyl, cycloalkylalkyl, cycloalkylalkenyl, heterocycle, (heterocycle)alkyl, or (heterocycle)alkenyl, optionally substituted;
Drug₂ is Drug₂-1 or Drug₂-2, 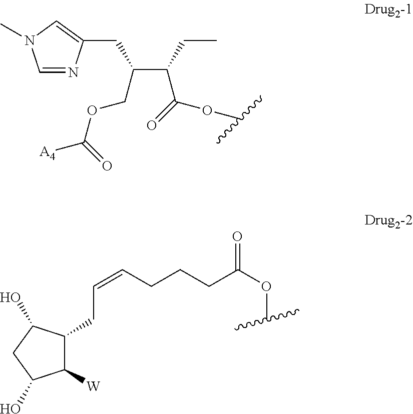
A₄ is alkyl, cycloalkyl, cycloalkylalkyl, or arylalkyl, and
W of Drug₂-2 is W-1, W-2, W-3, W-4, or W-5,

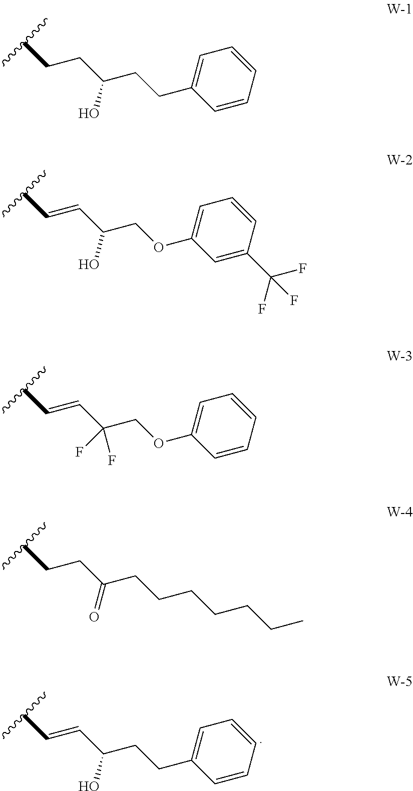

2. The compound of claim 1, wherein R₂ is R₂-1 or R₂-1.

3. The compound of claim 1, wherein n₁=n₂=1, or n₁=2 and n₂=1.

4. The compound of claim 1, wherein Drug₂ is Drug₂-1.

5. The compound of claim 1, wherein Drug₂ is Drug₂-2.

6. The compound of claim 1, wherein A₁ and A₂ are independently hydrogen, methyl, or ethyl; D is phenyl, pyridyl, (CH₂)ᵢCHA₃(CH₂)ⱼ, or (CH₂)ᵢC₆H₄(CH₂)ⱼ, where i and j are independently 0-4, and A₃ is hydrogen, alkyl, aryl, arylalkyl, heteroaryl, heteroarylalkyl, cycloalkyl, or cycloalkylalkyl.

7. The compound of claim 1, wherein the Link is Link-2, and D is CH₂ or CHCH₃.

8. The compound of claim 1, wherein the Link is Link-3, and D is CH₂, CH(CH₃), (CH₂)₃, (CH₂)₄, (CH₂)₅, or (CH₂)₂CHCH₃.

9. The compound of claim 1, wherein the Link is Link-4, A₁ is hydrogen, and A₂ is hydrogen or methyl.

10. The compound of claim 1, wherein Q is (CR₄R₅)ₙ₃; and n₃ is 1-3.

11. The compound of claim 1, wherein R₃, R₄ and R₅ are H, and R₈ is H, alkyl, arylalkyl, cycloalkyl, cycloalkylalkyl, or heterocycle.

12. The compound of claim 1, which is selected from the group consisting of: (Z)-2-(5-(((R)-3-(isoquinolin-5-ylamino)pyrrolidin-1-yl)methyl)-2-methylphenoxy)ethyl 7-((1R,2R,3R,5S)-3,5-dihydroxy-2-((R)-3-hydroxy-5-phenylpentyl)cyclopentyl)hept-5-enoate, Compound 1; (Z)-2-(5-(((R)-3-(isoquinolin-5-ylamino)pyrrolidin-1-yl)methyl)-2-methylphenoxy)ethyl 7-((1R,2R,3R,5S)-3,5-dihydroxy-2-((R,E)-3-hydroxy-4-(3-(trifluoromethyl)phenoxy)but-1-enyl)cyclopentyl)hept-5-enoate, Compound 2; (Z)-2-(5-(((R)-3-(isoquinolin-5-ylamino)pyrrolidin-1-yl)methyl)-2-methylphenoxy)ethyl 7-((1R,2R,3R,5S)-3,5-dihydroxy-2-(3-oxodecyl)cyclopentyl)hept-5-enoate, Compound 3; (Z)-2-(5-(((R)-3-(isoquinolin-5-ylamino)pyrrolidin-1-yl)methyl)-2-methylphenoxy)ethyl 7-((1R,2R,3R,5S)-2-((E)-3,3-difluoro-4-phenoxybut-1-enyl)-3,5-dihydroxycyclopentyl)hept-5-enoate, Compound 4; (5Z)-2-(5-(((R)-3-(1H-indazol-5-ylamino)piperidin-1-yl)methyl)-2-methylphenoxy)ethyl 7-((1R,2R,3R,5S)-3,5-dihydroxy-2-((S,E)-3-hydroxy-5-phenylpent-1-enyl)cyclopentyl)hept-5-enoate, Compound 5; (Z)-3-(2-(5-(((R)-3-(isoquinolin-5-ylamino)pyrrolidin-1-yl)methyl)-2-methylphenoxy)acetoxy)propyl 7-((1R,2R,3R,5S)-3,5-dihydroxy-2-((R)-3-hydroxy-5-phenylpentyl)cyclopentyl)hept-5-enoate, Compound 6; (Z)-3-(2-(3-(((S)-3-(1H-indazol-5-ylamino)piperidin-1-yl)methyl)phenoxy)acetoxy)propyl 7-((1R,2R,3R,5S)-3,5-dihydroxy-2-((R)-3-hydroxy-5-phenylpentyl)cyclopentyl)hept-5-enoate, Compound 7; (Z)-1-(N-(5-(((R)-3-(isoquinolin-5-ylamino)pyrrolidin-1-yl)methyl)-2-methylphenyl)ethylsulfonamido)ethyl 7-((1R,2R,3R,5S)-3,5-dihydroxy-2-((R)-3-hydroxy-5-phenylpentyl)cyclopentyl)hept-5-enoate, Compound 8; (Z)-1-(N-(3-(((S)-3-(1H-indazol-5-ylamino)piperidin-1-yl)methyl)phenyl)methylsulfonamido)ethyl 7-((1R,2R,3R,5S)-3,5-dihydroxy-2-((R)-3-hydroxy-5-phenylpentyl)cyclopentyl)hept-5-enoate, Compound 9; (Z)-1-(6-(((R)-3-(isoquinolin-5-ylamino)pyrrolidin-1-yl)methyl)-1H-indol-1-yl)ethyl 7-((1R,2R,3R,5S)-3,5-dihydroxy-2-((R)-3-hydroxy-5-phenylpentyl)cyclopentyl)hept-5-enoate, Compound 10; (Z)-1-(6-(((R)-3-(1H-indazol-5-ylamino)piperidin-1-yl)methyl)-1H-indol-1-yl)ethyl 7-((1R,2R,3R,5S)-3,5-dihydroxy-2-((R)-3-hydroxy-5-phenylpentyl)cyclopentyl)hept-5-enoate, Compound 11; (Z)-1-(3-(((S)-3-(1H-indazol-5-ylamino)piperidin-1-yl)methyl)benzylcarbamoyloxy)ethyl 7-((1R,2R,3R,5S)-3,5-dihydroxy-2-((R)-3-hydroxy-5-phenylpentyl)cyclopentyl)hept-5-enoate, Compound 12; and (2S,3R)-2-(5-(((R)-3-(isoquinolin-5-ylamino)pyrrolidin-1-yl)methyl)-2-methylphenoxy)ethyl 2-ethyl-4-(1-methyl-1H-imidazol-4-yl)-3-(propionyloxymethyl)butanoate, Compound 13.

13. A method of lowering intraocular pressure in a subject, comprising the steps of:
identifying a subject in need thereof; and
administering to the subject an effective amount of the compound of claim 1 to lower the intraocular pressure of the subject.

14. A method of treating allergic conjunctivitis, macular edema, macular degeneration, or blepharitis in a subject, comprising the steps of:
identifying a subject in need thereof; and
administering to the subject an effective amount of the compound of claim 1 to lower the intraocular pressure of the subject.
